(19) United States
(12) Patent Application Publication
(10) Pub. No.: US 2018/0355312 A1
(43) Pub. Date: Dec. 13, 2018

(54) METHODS, MEDIA AND PRODUCTS FOR CULTURING EMBRYOS

(71) Applicants: THE UNIVERSITY OF ADELAIDE, Adelaide, South Australia (AU); MONASH IVF GROUP LIMITED, Richmond, Victoria (AU)

(72) Inventors: Michelle Lane, Ironbank, South Australia (AU); Deirdre Zander-Fox, Semaphore, South Australia (AU)

(21) Appl. No.: 15/778,664

(22) PCT Filed: Nov. 24, 2016
(86) PCT No.: PCT/AU2016/051154

§ 371 Date: May 24, 2018

(30) Foreign Application Priority Data
Nov. 24, 2015 (AU) 2015904859

(51) Int. Cl. C12N 5/073; A01N 1/02

(52) U.S. Cl. CPC C12N 5/0604; C12N 2517/10; A01N 1/0221; A01N 1/0284

(57) ABSTRACT

The present invention relates to a method of culturing an embryo for implantation, the method comprising: incubating a pre-compaction stage embryo in a first embryo culture medium; adding a second medium to the first embryo culture medium to form a compounded embryo culture medium; and further incubating the embryo in the compounded embryo culture medium for implantation.

### PRIORITY CLAIM

This application claims priority to Australian Provisional Patent Application 2015904859 filed on 24 Nov. 2015, the content of which is hereby incorporated by reference.

### FIELD

The present disclosure relates to methods for culturing embryos, media for culturing embryos, compounding media and products for culturing embryos.

### BACKGROUND

Assisted reproduction technologies (ART) are a variety of reproductive technologies used in humans and animals that involve some form of treatment and/or intervention to achieve pregnancy. Some of these technologies involve the production of embryos in vitro and the subsequent transfer of the embryo to a recipient. Examples of such technologies include in vitro fertilization (IVF), intracytoplasmic sperm injection (ICSI) and cytoplasmic transfer.

In vitro fertilization involves the fertilization of an egg by sperm in vitro. The procedure is used in both humans and animals, such as domestic farm animals. In humans, the rate at which IVF is being used to achieve pregnancy is increasing. For example, in Australia approximately 1 in 6 couples utilise IVF to achieve pregnancy at a cost of more than $1 billion dollars per annum.

Although the first child born using in vitro fertilization (IVF) was born over 30 years ago, success rates after IVF are still low with more than 60% of all embryos created being deemed non-viable. This results in the need to undergo multiple attempts at IVF and to transfer multiple embryos to a patient to maximize pregnancy rates. This has resulted in an increase in multiple gestation pregnancies, which can result in significant complications to the mother and the babies, including the possibility of long term health risks.

Epidemiological studies on the outcomes following human IVF report that singleton babies born after IVF are smaller than matched controls conceived naturally. Recent data also continues to highlight the fact that the composition of embryo culture media may be involved in fetal programming.

Embryonic development is a highly complex process. Technology for the culture of human embryos has been primarily static since the late 1990's, and involves the use of sequential media to culture embryos. This sequential culturing technology aims to provide the developing embryo with the nutrients required at specific stages of development, based on the environment in the reproductive tract. Although this stage specific nutrient exposure is beneficial, embryos have to be washed extensively and moved to new medium, typically at around the 8-cell stage, as the nutrients and media components that are optimal for the early embryo are detrimental later in development and vice versa. Additionally, the waste product ammonium can build up in the media which can be toxic once it reaches a threshold concentration. Sequential media formulations can lead to toxic levels of ammonium, typically within 48 to 72 hours of culture. Therefore, all culture medium must be renewed before toxic thresholds are met, requiring the embryo to be removed from the cell media. Sequential media technologies also remove the embryo from an environment containing important autocrine and/or paracrine secreted factors which are beneficial for future development. Single-step systems have been used to culture embryos without requiring a medium change. However such methods are thought to impact embryo quality as they induce metabolic stress on the embryo by exposing the embryo to waste products and an unchanged carbohydrate profile.

Advancements in medium formulations over the last few years have focussed primarily on the reduction of in vitro induced stress, which have only resulted in incremental increases in pregnancy rates.

As can be seen, for a variety of reasons there is a need to improve embryo culture techniques.

### SUMMARY

The present disclosure relates to methods for culturing embryos, media for culturing embryos, compounding media and products for culturing embryos.

The present disclosure is predicated, at least in part, on the surprising determination that a high quality embryo for implantation can be cultured in a system which does not require the embryo to be transferred from a first embryo culture medium to a second embryo culture medium in a sequential manner.

Prior to the present disclosure, it was considered that the provision of high quality embryos required a transfer from a first culture medium which provided nutrients required for early embryo development to a second culture medium which provided nutrients required for later embryo development. An important aspect of this transfer is a wash of the embryo to remove components which are optimal for early development but detrimental for later development and also to remove waste ammonium from the culture. It was considered that not transferring and washing the embryo may cause metabolic stress caused by exposure to waste products and an unchanging carbohydrate profile.

Certain embodiments of the present disclosure provide a method of culturing an embryo for implantation, the method comprising:
incubating a pre-compaction stage embryo in a first embryo culture medium;
adding a second medium to the first embryo culture medium to form a compounded embryo culture medium; and
further incubating the embryo in the compounded embryo culture medium for implantation.

As used herein, the second medium/compounding medium is different to the first embryo culture medium. In other words the second medium/compounding medium comprises one or more component(s) which are not present in the first culturing medium and/or comprises one or more component(s) at a different concentration to the first culturing medium and/or does not comprise one or more component(s) which are present in the first culture medium. In one embodiment, the second medium/compounding medium comprises one or more component(s) which are not present in the first culturing medium.

Certain embodiments of the present disclosure provide a method of culturing an embryo for implantation, the method comprising use of a compounding medium added to a first culture medium to culture the embryo.

Certain embodiments of the present disclosure provide a method of culturing an embryo for implantation, the method comprising culturing the embryo in one or more culture media that does not substantially comprise ethylenediaminetetraacetic acid (EDTA) and/or a salt thereof.

Certain embodiments of the present disclosure provide a method of culturing an embryo for implantation, the method comprising culturing the embryo in one or more culture media comprising acetyl-carnitine (and/or an acceptable salt and/or derivative thereof).

Certain embodiments of the present disclosure provide a method of assisted reproduction, the method comprising culturing an embryo for implantation using a method as described herein and implanting the embryo into a subject.

Certain embodiments of the present disclosure provide an embryo culture medium comprising acetyl-carnitine (and/or an acceptable salt and/or derivative thereof).

Certain embodiments of the present disclosure provide an embryo culture medium comprising pyruvate and/or lactate and substantially no ethylenediaminetetraacetic acid (EDTA) and/or a salt thereof.

Certain embodiments of the present disclosure provide a method of culturing an embryo, the method comprising culturing the embryo in a medium as described herein.

Certain embodiments of the present disclosure provide a compounding medium for addition to an embryo culture medium, the medium comprising a glucose concentration of at least 3.5 mM glucose and less lactate or pyruvate than the first embryo culture medium. In certain embodiments the compounding medium comprises less lactate and or pyruvate than the first embryo culture medium. In certain embodiments the compounding medium comprises 10%, 20%, 30%, 40%, 50%, 60%, 70%, 80%, or 90% of the lactate or pyruvate content compared to the first embryo culture medium. In certain embodiments, the compounding medium comprises substantially no lactate and/or pyruvate.

Certain embodiments of the present disclosure provide a compounding medium for addition to an embryo culture medium, the medium comprising a glucose concentration of at least 3.5 mM glucose and substantially no lactate.

Certain embodiments of the present disclosure provide a compounding medium for addition to an embryo culture medium, the medium comprising a glucose concentration of at least 3.5 mM glucose and substantially no pyruvate.

In certain embodiments the compounding medium comprises more glucose than the first embryo culture medium. In certain embodiments the compounding medium may comprise 150%, 200%, 300%, 400%, 500%, 600%, 700%, 800%, 900% or 1000% of the glucose content compared to the first embryo culture medium.

Certain embodiments of the present disclosure provide a method of culturing an embryo for implantation, the method comprising:
incubating a pre-compaction embryo in an embryo culture medium as described herein;
adding a compounding medium as described herein to the first embryo culture medium to form a compounded embryo culture medium; and
further incubating the embryo in the compounded embryo culture medium for implantation.

Certain embodiments of the present disclosure provide a combination product comprising:
(i) an embryo culture medium as described herein; and
(ii) a compounding medium as described herein.

Certain embodiments of the present disclosure provide a kit for performing a method as described herein.

Certain embodiments of the present invention provide a kit comprising:
(i) an embryo culture medium as described herein; and/or
(ii) a compounding medium as described herein.

Certain embodiments of the present disclosure provide a non-human animal produced using a method as described herein.

Certain embodiments of the present disclosure provide a method of vitrification of an embryo, the method comprising:
incubating a pre-compaction stage embryo in a first embryo culture medium;
adding a second medium to the first embryo culture medium to form a compounded embryo culture medium;
further incubating the embryo in the compounded embryo culture medium; and
freezing the embryo incubated in the compounded embryo medium.

Certain embodiments of the present disclosure provide a vitrified embryo produced using a method of vitrification as described herein.

Certain embodiments of the present disclosure provide a non-human animal produced using a method as described herein.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows the results of various parameters measured for embryos cultured in either the compounding culture media (CCM) system described herein or commercial products. Panel A shows implantation rates (implantation sites/embryos transferred), Panel B shows fetal development rates (fetuses/embryos transferred) and Panel C shows fetuses/implantation rates for embryos cultured in either CCM or commercial products. Mouse embryos were cultured and transferred to pseudopregnant recipients and on day 18 of pregnancy implantation and fetal development assessed. Different superscripts are significantly different (P<0.05). CCM performs equal to or better than commercial media products.

FIG. 2 shows fetal weights at day 18 of pregnancy. Panel A shows Sydney IVF media from COOK, Panel B shows Vitrolife G1/G2 media and Panel C shows Global media. Mouse embryos were cultured and transferred to pseudopregnant recipients and on day 18 of pregnancy implantation and fetal development assessed. Different superscripts are significantly different (P<0.05). CCM results in heavier or equivalent foetuses compared to commercial media products.

FIG. 3 shows placental weights at day 18 of pregnancy. Mouse embryos were cultured and transferred to pseudopregnant recipients and on day 18 of pregnancy implantation and fetal development assessed. Different superscripts are significantly different (P<0.05). CCM results in heavier or equivalent placentas compared to commercial media products.

FIG. 4 shows mouse embryo development in Sequential Culture with (G-1™/G-2™) and Compound Culture (G-compounding media).

FIG. 5 shows mouse Day 5 blastocyst cell number in Sequential Culture with (G-1™/G-2™) and Compound Culture (G-compounding media) (Data shown is the mean cell number and 95% confidence interval).

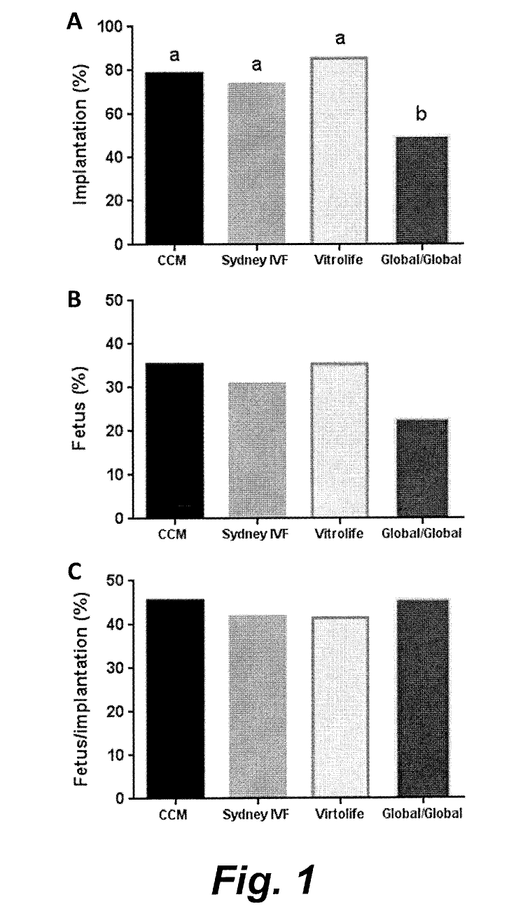

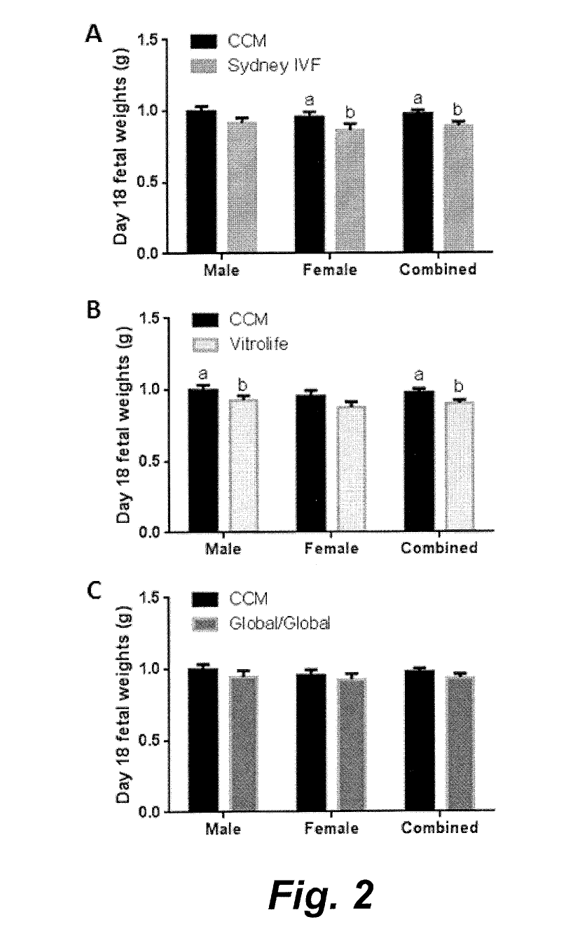

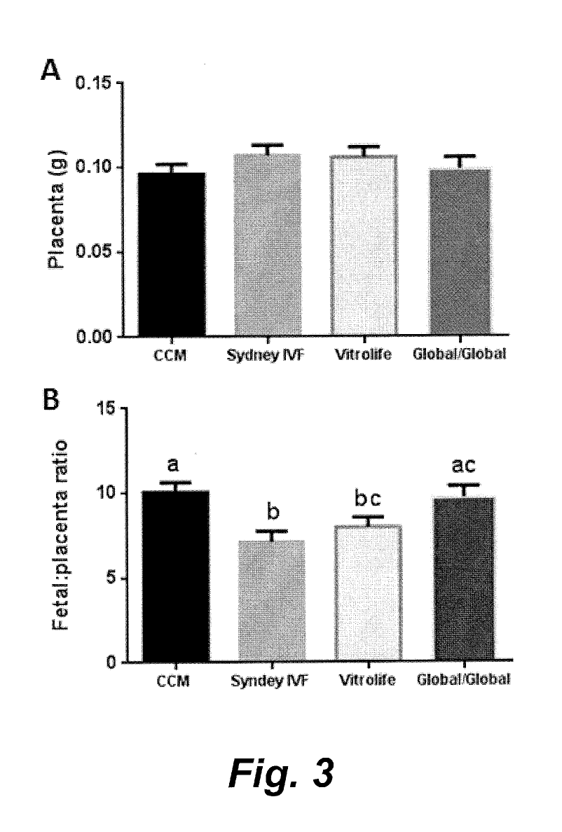

### DETAILED DESCRIPTION

The present disclosure relates to methods for culturing embryos, media for culturing embryos, compounding media and products for culturing embryos.

The present disclosure is based on the determination that a compounding system may be utilised to culture embryos, rather than the use of a sequential media system for culturing embryos, and that the use of a compounding media system provides advantages not only to the developing embryo but also economic and commercial advantages in the way that embryos may be cultured.

Certain embodiments of the present disclosure are directed to methods and products that have one or more combinations of advantages. For example, some advantages of some embodiments disclosed herein include one or more of the following: to provide a compounding embryo culture system; to provide an embryo culturing system which reduces handling of embryos; to provide an embryo culture system that eliminates the need to remove embryos from their original culture medium; to provide an embryo culture system that eliminates the need to wash embryos during culturing; to provide an embryo culturing system for assisted reproduction technologies for use in humans and/or animals; to provide alternative embryo culturing systems for implantation; to provide an improved media culturing system for embryos; to increase cell number in the embryo at the blastocyst stage, e.g. when measured at 5 to 6 days of culturing in a system according to the present disclosure; to provide an embryo culture system that assists in maintaining ammonium levels below toxic levels throughout the culture period; to preserve autocrine and/or paracrine factors produced by an embryo in the culture environment; to provide an embryo culture system that assists in preserving the gradient of pyruvate concentration during culturing; to provide an embryo culture system that assists in preserving the gradient of lactate concentration during culturing; to provide an embryo culture system that assists in preserving the gradient of glucose concentration during culturing; to provide an embryo culture system that assists in maintaining the switch from non-essential amino acids to essential amino acids during embryonic development; to provide an embryo culture system that is compatible with time lapse technology and/or metabolic viability assessments; to address one or more problems and/or to provide one or more advantages, or to provide a commercial alternative. Other advantages of certain embodiments of the present disclosure are also disclosed herein.

Certain embodiments of the present disclosure provide a method of culturing an embryo.

In certain embodiments, the methods as described herein are used for the purposes of culturing an embryo for implantation. Implantation refers to the process whereby an embryo is placed into an in vivo environment where it may eventually develop into a fetus.

In certain embodiments, the methods of culturing an embryo for implantation as described herein are used in assisted reproductive technologies. Examples of assisted reproductive technologies include in vitro fertilization (IVF), in vitro maturation of oocytes, intracytoplasmic sperm injection (ICSI) and cytoplasmic transfer.

In certain embodiments, the methods as described herein are used in assisted reproductive technologies. Examples of assisted reproductive technologies include in vitro fertilization (IVF), in vitro maturation of oocytes, intracytoplasmic sperm injection (ICSI) and cytoplasmic transfer.

In certain embodiments, the methods as described herein are used in assisted reproductive technologies in humans. In certain embodiments, the methods are used as part of the IVF process in humans.

In certain embodiments, the methods as described herein in assisted reproductive technologies in animals. Examples of assisted reproductive technologies include in vitro fertilization (IVF), in vitro maturation of oocytes, intracytoplasmic sperm injection (ICSI) and cytoplasmic transfer.

Certain embodiments of the present disclosure provide a method of culturing an embryo for implantation, the method comprising:
incubating a pre-compaction stage embryo in a first embryo culture medium;
adding a second medium to the first embryo culture medium to form a compounded embryo culture medium; and
further incubating the embryo in the compounded embryo culture medium for implantation.

In certain embodiments, the embryo is a human embryo. In certain embodiments, the embryo is an embryo from a human subject suffering from, or susceptible to, reduced fertility, a disease or condition associated with dysfunctional ovulation (such as polycystic ovarian syndrome or hyperprolactinemia), damaged fallopian tubes, presence of adhesions, or other disease or condition that results in reduced fertility.

In certain embodiments, the method is used in an assisted reproductive technology in a human. Examples of assisted reproductive technologies include in vitro fertilization (IVF), in vitro maturation of oocytes, intracytoplasmic sperm injection (ICSI) and cytoplasmic transfer. In certain embodiments, the method is used as part of the IVF process in a human.

In certain embodiments, the embryo is an animal embryo.

In certain embodiments, the embryo is a mammalian embryo such as an embryo from a livestock animal (such as a horse, a cow, a sheep, a goat, a pig, a camel), a domestic animal (such as a dog or a cat) and other types of animals such as non-human primates, rabbits, mice and laboratory animals. Other types of animals are contemplated.

In certain embodiments, the method is used in an assisted reproductive technology in an animal. Examples of assisted reproductive technologies include in vitro fertilization (IVF), in vitro maturation of oocytes, intracytoplasmic sperm injection (ICSI) and cytoplasmic transfer. In certain embodiments, the method is used as part of the IVF process in an animal. For example, the methods described herein may be used in Bos Taurus or Bos Indicus for assisted reproductive purposes.

In certain embodiments, the incubating of the pre-compaction embryo in the first embryo culture medium comprises a period of time sufficient for the embryo to form a post-compaction embryo. Incubating and culturing conditions are described herein.

In certain embodiments, the incubating of the pre-compaction embryo in the first embryo culture medium comprises a period of time in the range from 24 to 72 hours.

In certain embodiments, the incubating of the pre-compaction embryo in the first embryo culture medium comprises a period of time in the range from 24 to 72, 36 to 72, 48 to 72, 24 to 60, 36 to 60, 48 to 60, 48 to 72, or 60 to 72 hours.

Suitably, the first embryo culture medium may be any culture medium which is capable of supporting the embryo up to day 3 (around the 8-cell stage). In particular, the first culture medium may be any culture medium which is capable of supporting the pre-compaction embryo (eg to the post-compaction stage). Such a medium contains carbohydrates, amino acids and chelators to support the early embryo.

The first culture medium may be a commercially available culture medium capable of supporting the embryo up to day 3 (around 8-cell stage). An illustrative example of a suitable, commercially available medium is the G-1™ medium provided by Vitrolife, the complete formulation for which is shown in Table A below:

TABLE A

| | A | | | |
|---|---|---|---|---|
| | Most | | B | |
| | Preferred | | Preferred | |
| Component | Concentration | | Range | |
| NaCl | 90.08 | | 80.0-100 | |
| KCl | 5.5 | | 3.5-7.5 | |
| NaH₂PO₄ | 0.25 | | 0.05-1.5 | |
| MgSO₄ | 1 | | 0.2-2.0 | |
| NaHCO₃ | 25 | | 15.0-30 | |
| CaC1₂ | 1.8 | | 0.8-2.8 | |
| Glucose | 0.5 | | 0.05-5.0 | |
| NaLactate | 10.5 | | 5.0-20 | |
| (L-isomer) | | | | |
| NaPyruvate | 0.32 | | 0.1-1.0 | |
| Alanine | 0.1 | | 0.01-0.5 | |
| Asparate | 0.1 | | 0.01-0.5 | |
| Asparagine | 0.1 | | 0.01-0.5 | |
| Glutamate | 0.1 | | 0.01-0.5 | |
| Alanyl-Glutamine | 0.5 | | 0.1-1.0 | |
| Glycine | 0.1 | | 0.01-0.5 | |
| Proline | 0.1 | | 0.01-0.5 | |
| Serine | 0.1 | | 0.01-0.5 | |
| Taurine | 0.1 | | 0.01-10.0 | |
| EDTA | 0.01 | | 0.005-0.20 | |
| HAS | 5 | mg/ml | 1-10.0 | mg/ml |
| Hyaluronate | 0.1 | mg/ml | 0.02-0.5 | mg/ml |

The concentrations in this table are provided in mM, unless otherwise indicated.

In certain embodiments, the incubating of the embryo in the compounded embryo culture medium comprises a period of time sufficient for the embryo to form a morula or blastocyst.

In certain embodiments, the first embryo culture medium does not comprise EDTA. In certain embodiments, the first embryo culture medium does not substantially comprise EDTA. The term “substantially no EDTA” as used herein means 0.01 mM or less EDTA.

In certain embodiments, the first embryo culture medium comprises acetyl-carnitine, and/or an acceptable salt and/or derivative thereof.

In certain embodiments, the first embryo culture medium comprises an effective amount of acetyl-carnitine, and/or an acceptable salt and/or derivative thereof. In certain embodiments, the concentration of acetyl carnitine (and/or an acceptable salt and/or derivative thereof) in the first medium comprises 5 μM to 1 mM, 10 μM to 1 mM, or 40 μM to 1 mM.

In one embodiment, the first embryo culture medium comprises 5 μM to 50 μM, 5 μM to 20 μM, 5 μM to 15 μM, or about 10 μM of acetyl carnitine (and/or an acceptable salt and/or derivative thereof).

In certain embodiments, the first embryo culture medium comprises acetyl-carnitine (and/or an acceptable salt and/or derivative thereof) and the medium does not substantially comprise EDTA. The term “substantially no EDTA” means 0.01 mM or less EDTA.

In certain embodiments, the first embryo culture medium comprises pyruvate, such as sodium pyruvate.

In certain embodiments, the first embryo culture medium comprises an effective amount of pyruvate. In certain embodiments, the first embryo culture medium comprises greater than 0.1 mM pyruvate, or greater than 0.20 mM pyruvate.

In certain embodiments, the first embryo culture medium comprises greater than 0.25 mM, greater than 0.30 mM, or 0.32 or greater mM pyruvate.

In one embodiment, the first embryo culture medium comprises greater than or equal to 0.32 mM pyruvate.

In certain embodiments, the first embryo culture medium comprises aspartate/aspartic acid.

In certain embodiments, the first embryo culture medium comprises an effective amount of aspartate. In certain embodiments, the first embryo culture medium comprises greater than 0.01 mM aspartate, greater than 0.10 mM aspartate, greater than 0.15 mM, greater than 0.20 mM, greater than 0.25 mM, greater than 0.30 mM, or equal to or greater than 0.32 mM aspartate.

In one embodiment, the first embryo culture medium comprises greater than or equal to 0.32 mM aspartate.

In certain embodiments, the first embryo culture medium comprises glycine/glycinate.

In certain embodiments, the first embryo culture medium comprises an effective amount of glycine. In certain embodiments, the first embryo culture medium comprises greater than 0.01 mM glycine, greater than 0.10 mM glycine, greater than 0.15 mM, greater than 0.20 mM, greater than 0.25 mM, greater than 0.30 mM, or equal to or greater than 0.32 mM.

In one embodiment, the first embryo culture medium comprises greater than or equal to 0.32 mM glycine.

In certain embodiments, the first embryo culture medium comprises one or more of the following components: at least 0.05 mM glucose or at least 0.1 mM glucose; at least 2 mM lactate or greater than 5 mM lactate; at least 0.1 mM pyruvate or at last 0.3 mM pyruvate; at least 0.01 mM apartate or at least 0.1 mM aspartate; and/or at least 0.01 mM glycine or at least 0.1 mM glycine; and optionally at least 0.12 mM acetyl-carnitine and/or at least 0.1 mM glutamine and/or at least 0.1 mM alanyl-glutamine.

In certain embodiments, the first embryo culture medium comprises a dipeptide. For example, the first embryo culture medium may comprise glycyl-L-glutamine. In certain embodiments, the use of a dipeptide replaces the use of one or more amino acids.

In certain embodiments, the first embryo medium comprises one or more of the components as substantially as described in Table 1 or Table A herein.

In certain embodiments, the first embryo medium is substantially as described in Table 1 or Table A herein.

In one embodiment the first embryo medium may comprise a protein source. The protein source may be albumin or synthetic serum (e.g. at a concentration of 5 to 20% w/v or v/v respectively). Suitable sources for protein supplementation include human serum, human cord serum (HCS), human serum albumin (HSA), fetal calf serum (FCS) or bovine serum albumin (BSA).

In one embodiment the first embryo medium may comprise a growth factor.

In certain embodiments, the first embryo culture medium comprises albumin. Albumin may be from any source known in the art. The albumin may be a recombinant albumin. The albumin may be human serum albumin (HSA). The first embryo culture medium may comprise albumin at an effective concentration. The first embryo culture medium may comprise albumin at a concentration of about 2.5 to 10 mg/ml. The concentration of albumin may be about 3 to 9 mg/ml, about 4 to 8 mg/ml or about 5 to 7 mg/l. The concentration of albumin in the first embryo culture medium may be from about 2.5 to 5 mg/ml. In one embodiment the concentration of albumin in the first embryo culture medium may be about 2.5, 3, 4, 5, 6, 7, 8, 9 or 10 mg/ml. In one embodiment the concentration of albumin in the first embryo culture medium may be about 5 mg/ml. In another embodiment the concentration of albumin in the first embryo culture medium may be about 2.5 mg/ml. In one embodiment the first embryo culture medium comprises human serum albumin at a concentration of about 5 mg/ml. In one embodiment the first embryo culture medium comprises recombinant albumin at a concentration of about 2.5 mg/ml.

The term “second medium” as used herein refers to a compounding medium that is added to a first embryo culture medium to form a “compounded medium”. Accordingly, the terms “second medium” and “compounding medium” as used herein refer to the same medium. It will also be appreciated that the term “compounded medium” refers to the medium resulting from the addition of the second medium to the first embryo culture medium.

An illustrative example of the formulation of the compounding medium (i.e. the second medium/compounding medium) is shown in Table B below:

TABLE B

| | A | | | |
|---|---|---|---|---|
| | Most | | B | |
| | Preferred | | Preferred | |
| Component | Concentration | | Range | |
| NaCl | 90.08 | | 80.0-100 | |
| KCl | 5.5 | | 3.5-7.5 | |
| NaH₂PO₄ | 0.25 | | 0.05-1.5 | |
| MgSO₄ | 1 | | 0.2-4.0 | |
| NaHCO₃ | 25 | | 15-30.0 | |
| CaCl₂ | 1.8 | | 0.8-2.8 | |
| Glucose | 5.8 | | 1.0-11.0 | |
| NaLactate (L-isomer) | 1.24 | | 0.1-10.0 | |
| NaPyruvate | 0 | | 0-1.0 | |
| Alanine | 0.1 | | 0.01-0.5 | |
| Aspartate | 0.1 | | 0.01-0.5 | |
| Asparagine | 0.1 | | 0.01-0.5 | |
| Glutamate | 0.1 | | 0.01-0.5 | |
| Alanyl-Glutamine | 1.5 | | 0.01-3.0 | |
| Glycine | 0.1 | | 0.01-0.5 | |
| Proline | 0.1 | | 0.01-0.5 | |
| Serine | 0.1 | | 0.01-0.5 | |
| L-Arginine-HCl | 1.2 | | 0.1-2.4 | |
| L-Cystine 2HCl | 0.2 | | 0.05-0.5 | |
| L-Histidine-HCl—H₂O | 0.4 | | 0.1-0.8 | |
| L-Isoleucine | 0.8 | | 0.1-1.6 | |
| L-Leucine | 0.8 | | 0.1-1.6 | |
| L-Lysine-HCl | 0.8 | | 0.1-1.6 | |
| L-Methionine | 0.2 | | 0.05-0.5 | |
| L-Phenylalanine | 0.4 | | 0.1-0.8 | |
| L-Threonine | 0.8 | | 0.1-1.6 | |
| L-Tryptophan | 1.0 | | 0.1-1.8 | |
| L-Tyrosine 2Na | 0.4 | | 0.1-0.8 | |
| L-Valine | 0.8 | | 0.1-1.6 | |
| D-Ca Pantothenate | 0.004 | | 0.001-0.008 | |
| Choline Chloride | 0.014 | | 0.003-0.03 | |
| Folic Acid | 0.0046 | | 0.001-0.009 | |
| i-Inositol | 0.0222 | | 0.005-0.04 | |
| Niacinamide | 0.0164 | | 0.004-0.04 | |
| Pyridoxal HCl | 0.0098 | | 0.002-0.02 | |
| Riboflavin | 0.0006 | | 0.0001-0.002 | |
| Thiamine HCl | 0.006 | | 0.001-0.02 | |
| HSA | 5 | mg/ml | 1-10 | mg/ml |
| Hyaluronate | 0.1 | mg/ml | 0.02-0.5 | mg/ml |

The concentrations in this table are provided in mM, unless otherwise indicated.

In one embodiment the second medium may comprise a protein source. The protein source may be albumin or synthetic serum (e.g. at a concentration of 5 to 20% w/v or v/v respectively). Suitable sources for protein supplementation include human serum, human cord serum (HCS), human serum albumin (HSA), fetal calf serum (FCS) or bovine serum albumin (BSA).

In one embodiment the second medium may comprise a growth factor.

In certain embodiments, the second medium comprises albumin. Albumin may be from any source known in the art. The albumin may be a recombinant albumin. The albumin may be human serum albumin (HSA). The second medium may comprise albumin at an effective concentration. The second medium may comprise albumin at a concentration of about 2.5 to 10 mg/ml. The concentration of albumin may be about 3 to 9 mg/ml, about 4 to 8 mg/ml or about 5 to 7 mg/l. The concentration of albumin in the second medium may be from about 2.5 to 5 mg/ml. In one embodiment the concentration of albumin in the second medium may be about 2.5, 3, 4, 5, 6, 7, 8, 9 or 10 mg/ml. In one embodiment the concentration of albumin in the second medium may be about 5 mg/ml. In another embodiment the concentration of albumin in the second medium may be about 2.5 mg/ml. In one embodiment the second medium comprises human serum albumin at a concentration of about 5 mg/ml. In one embodiment the second medium comprises recombinant albumin at a concentration of about 2.5 mg/ml.

In certain embodiments the first medium, second medium or compounded medium comprises antioxidants. The medium may comprise any suitable antioxidants known in the art. Suitable antioxidants which may be used in the second medium include acetyl-carnitine, lipoic acid or a derivative thereof, acetyl-cysteine, ascorbic acid and 2-mercaptoethanol.

In one embodiment acetyl-carnitine is present in the medium of the present disclosure, or for use in the present disclosure, at a concentration of about 5 μM to about 1 mM.

In one embodiment acetyl-carnitine is present in the medium of the present disclosure, or for use in the present disclosure, at 5 μM to about 50 μM.

In another embodiment acetyl-carnitine is present in the medium of (or for use in) the present disclosure at a concentration from about 5 μM to about 15 μM. In another embodiment the acetyl-carnitine is present in the medium of (or for use in) the present disclosure at a concentration of about 10 μM.

In one embodiment lipoic acid or a derivative thereof is present in the medium of (or for use in) the present disclosure at a concentration of about 2.5 μM to about 40 μM, e.g. 5 μM to about 20 μM.

In another embodiment lipoic acid or a derivative thereof is present in the medium of (or for use in) the present disclosure at a concentration of about 2.5 μM to about 10 μM, e.g. 5 μM to about 10 μM.

In one embodiment lipoic acid or a derivative thereof is present in the medium of (or for use in) the present disclosure at a concentration of about 5 μM.

In one embodiment the medium according to the present disclosure comprises acetyl-cysteine at a concentration of about 5 to about 50 μM.

In one embodiment acetyl-cysteine is present in the medium of (or for use in) the present disclosure at a concentration of about 5 to about 20 μM.

In another embodiment acetyl-cysteine is present in the medium of (or for use in) the present disclosure at a concentration of about 5 μM to about 15 μM.

In one embodiment acetyl-cysteine is present in the medium of (or for use in) the present disclosure at a concentration of about 10 μM.

In certain embodiments, the medium may comprise one or more of acetyl-carnitine, lipoic acid or a derivative thereof and acetyl-cysteine.

In one embodiment the medium comprises acetyl-carnitine and lipoic acid or a derivative thereof. In one embodiment the medium comprises acetyl-carnitine at a concentration of about 5 to about 50 μM; and lipoic acid or a derivative thereof at a concentration of about 2.5 to about 40 μM. In another embodiment the medium comprises acetyl-carnitine, lipoic acid or a derivative thereof and acetyl-cysteine. In another embodiment the medium comprises acetyl-carnitine at a concentration of about 5 to about 50 μM; lipoic acid or a derivative thereof at a concentration of about 2.5 to about 40 μM; and acetyl-cysteine at a concentration of about 5 to about 50 μM.

In one embodiment the medium comprises acetyl-carnitine and acetyl-cysteine. In one embodiment the medium comprises acetyl-carnitine at a concentration of about 5 to about 50 μM and acetyl-cysteine at a concentration of about 5 to about 50 μM.

In one embodiment the medium comprises lipoic acid or a derivative thereof and acetyl-cysteine. In one embodiment the medium comprises lipoic acid or a derivative thereof at a concentration of about 2.5 to about 40 μM and acetyl-cysteine at a concentration of about 5 to about 50 μM.

In one embodiment the present disclosure relates to the use of lipoic acid or a derivative thereof. The term lipoic acid includes α-lipoic acid. This compound can be any racemic form e.g. (±)-1,2-Dithiolane-3-pentanoic acid, (R)-5-(1,2-dithiolane-3-yl)pentanoic acid or (S)-1,2-Dithiolane-3-pentanoic acid. The lipoic acid or derivative thereof may be added as a mixture of enantiomeric forms, or as a single enantiomer. In the latter case, the R-enantiomer has been found to be more biologically active. One derivative of lipoic acid for use in the present invention is lipoate. Lipoate is a salt or ester derivative of lipoic acid. A further derivative of lipoic acid includes methylated lipoic acid. The term “derivative” as used herein in relation to lipoic acid includes biologically active amphiphilic disulfide/thiotic molecules that have essentially equivalent physiological properties as lipoic acid.

The term “acetyl-cysteine” as used herein may be N-acetyl-L-cysteine (NAC) (e.g. unmodified NAC), or a derivative thereof, such as N-acetylcysteine-amide (NACA). In one embodiment the term “acetyl-cysteine” as used herein means N-acetyl-L-cysteine (NAC) (e.g. unmodified NAC).

The term acetyl-carnitine may be referred to as acetyl-L-carnitine.

In certain embodiments the ratio of the volume of the first embryo culture medium to the second medium to provide the compounded medium is 10:1 i.e. 10 parts first embryo culture medium to 1 part second medium. In other embodiments the ratio of the volume of the first embryo culture medium to the second medium may be 9:1, 8:1, 7:1, 6:1, 5:1, 4:1, 3:1, 2:1; 1:1, 1:2, 1:3, 1:4, 1:5, 1:6, 1:7, 1:8, 1:9 or 1:10. In one embodiment the ratio of the volume of the first embryo culture medium to the second medium is 1:1.

The compounded medium (which is generated following the addition of the second medium/compounding medium to the first embryo culture medium) is capable of supporting the embryo beyond day 3 (8-cell stage). Accordingly, the compounded medium is capable of supporting the embryo from day 3 (8-cell stage) to the blastocyst stage prior to implantation. Such a medium contains carbohydrates, amino acids and vitamins to support the later stage embryo.

An illustrative example of the final formulation of the compounded culture medium (i.e. following the addition of the second medium/compounding medium to the first embryo culture medium) is shown in Table C below:

TABLE C

| | A | | | |
|---|---|---|---|---|
| | Most | | B | |
| | Preferred | | Preferred | |
| Component | Concentration | | Range | |
| NaCl | 90.08 | | 80.0-100 | |
| KCl | 5.5 | | 3.5-7.5 | |
| NaH₂PO₄ | 0.25 | | 0.05-1.5 | |
| MgSO₄ | 1 | | 0.2-3.0 | |
| NaHCO₃ | 25 | | 15-30.0 | |
| CaCl₂ | 1.8 | | 0.8-2.8 | |
| Glucose | 3.15 | | 0.53-8.0 | |
| NaLactate (L-isomer) | 5.87 | | 2.55-15.0 | |
| NaPyruvate | 0.16 | | 0.05-1.0 | |
| Alanine | 0.1 | | 0.01-0.5 | |
| Aspartate | 0.1 | | 0.01-0.5 | |
| Asparagine | 0.1 | | 0.01-0.5 | |
| Glutamate | 0.1 | | 0.01-0.5 | |
| Alanyl-Glutamine | 1 | | 0.06-2.0 | |
| Glycine | 0.1 | | 0.01-0.5 | |
| Proline | 0.1 | | 0.01-0.5 | |
| Serine | 0.1 | | 0.01-0.5 | |
| L-Arginine-HCl | 0.6 | | 0.05-1.2 | |
| L-Cystine 2HCl | 0.1 | | 0.025-0.25 | |
| L-Histidine-HCl—H₂O | 0.2 | | 0.05-0.4 | |
| L-Isoleucine | 0.4 | | 0.05-0.8 | |
| L-Leucine | 0.4 | | 0.05-0.8 | |
| L-Lysine-HCl | 0.4 | | 0.05-0.8 | |
| L-Methionine | 0.1 | | 0.025-0.25 | |
| L-Phenylalanine | 0.2 | | 0.05-0.4 | |
| L-Threonine | 0.4 | | 0.05-0.8 | |
| L-Tryptophan | 0.5 | | 0.05-0.9 | |
| L-Tyrosine 2Na | 0.2 | | 0.05-0.4 | |
| L-Valine | 0.4 | | 0.05-0.8 | |
| D-Ca Pantothenate | 0.002 | | 0.0005-0.004 | |
| Choline Chloride | 0.007 | | 0.0015-0.015 | |
| Folic Acid | 0.0023 | | 0.0005-0.0045 | |
| i-Inositol | 0.0111 | | 0.0025-0.02 | |
| Niacinamide | 0.0082 | | 0.002-0.02 | |
| Pyridoxal HCl | 0.0049 | | 0.001-0.01 | |
| Riboflavin | 0.0003 | | 0.00005-0.001 | |
| Thiamine HCl | 0.003 | | 0.0005-0.01 | |
| Taurine | 0.05 | | 0.005-5 | |
| EDTA | 0.005 | | 0.0025-0.1 | |
| HSA | 5 | mg/ml | 1-10 | mg/ml |
| Hyaluronate | 0.1 | mg/ml | 0.02-0.5 | mg/ml |

The concentrations in this table are provided in mM, unless otherwise indicated.

The second medium/compounding medium as described herein is formulated such that the combination of a first embryo culture medium and second medium as defined herein produces a compounded medium as defined herein.

In other words, combining a first culture medium with a second medium/compounding medium produces a compounded medium which is capable of supporting the embryo beyond day 3 (8-cell stage).

In one embodiment the compounded medium may comprise a protein source. The protein source may be albumin or synthetic serum (e.g. at a concentration of 5 to 20% w/v or v/v respectively). Suitable sources for protein supplementation include human serum, human cord serum (HCS), human serum albumin (HSA), fetal calf serum (FCS) or bovine serum albumin (BSA).

In certain embodiments, the compounded medium comprises albumin. Albumin may be from any source known in the art. Suitably the albumin may be a recombinant albumin. Suitably the albumin may be human serum albumin (HSA). The compounded medium may comprise albumin at an effective concentration. The compounded medium may comprise albumin at a concentration of about 2.5 to 10 mg/ml. In one embodiment, the concentration of albumin may be about 3 to 9 mg/ml, about 4 to 8 mg/ml or about 5 to 7 mg/l. In one embodiment, the concentration of albumin in the compounded medium may be from about 2.5 to 5 mg/ml. In one embodiment the concentration of albumin in the compounded medium may be about 2.5, 3, 4, 5, 6, 7, 8, 9 or 10 mg/ml. In one embodiment the concentration of albumin in the compounded medium may be about 5 mg/ml. In another embodiment the concentration of albumin in the compounded medium may be about 2.5 mg/ml. In one embodiment the compounded medium comprises human serum albumin at a concentration of about 5 mg/ml. In one embodiment the compounded medium comprises recombinant albumin at a concentration of about 2.5 mg/ml.

In certain embodiments, the second medium comprises no or substantially no pyruvate or lactate. In certain embodiments, the second medium comprises substantially no pyruvate. The term “substantially no pyruvate” as used herein means less than 0.01 mM pyruvate is in the second medium.

In certain embodiments, the second medium comprises no or substantially no lactate. The term “substantially no lactate” as used herein means less than 0.1 mM lactate is in the second medium.

In one embodiment the second medium comprises less than 10 mM, suitably less than 0.2 mM lactate. In one embodiment the second medium comprises less than or equal to 1.24 mM lactate.

In certain embodiments, the second medium comprises substantially no acetyl-carnitine or EDTA.

In certain embodiments, the second medium comprises no or substantially no acetyl-carnitine. The term “substantially no acetyl-carnitine” as used herein means 0.01 mM or less acetyl-carnitine is in the second medium.

In certain embodiments, the second medium comprises no or substantially no EDTA. The term “substantially no EDTA” as used herein means that 0.01 mM or less EDTA is in the second medium.

In certain embodiments, the second medium comprises no or substantially no glutamine and/or alanyl-glutamine. In certain embodiments, the second medium comprises no or substantially no glutamine. The term “substantially no glutamine” as used herein means 0.01 mM or less glutamine is in the second medium.

In certain embodiments, the second medium comprises no or substantially no alanyl-glutamine. The term “substantially no alanyl-glutamine” as used herein means 1.5 mM or less alanyl-glutamine is in the second medium.

In certain embodiments, the second medium comprises a glucose concentration of at least 1 mM glucose, or at least 5 mM glucose.

In certain embodiments, the second medium comprises one or more of the following components: at least 1 mM glucose, at least 0.01 mM aspartate, and no or substantially no glycine. The term “substantially no glycine” as used herein means that 0.01 mM or less glycine is in the second medium.

In certain embodiments, the compounded embryo culture medium comprises one or more of the following components: at least 0.05 mM glucose or at least 3 mM glucose; less than 15 mM lactate or less than 6 mM lactate; less than 1.0 mM pyruvate or less than 0.20 mM pyruvate; at least 0.01 mM aspartate; and/or at least 0.01 mM glycine, at least 0.05 mM glycine; optionally at least 0.06 mM glutamine or at least 0.1 mM glutamine; and/or optionally at least 0.06 mM alanyl-glutamine or at least 0.1 mM alanyl-glutamine; and/or optionally at least 0.01 mM acetyl-carnitine.

In certain embodiments, the second medium comprises a dipeptide. For example, the second medium may comprise glycyl-L-glutamine. In certain embodiments, the use of a dipeptide replaces the use of one or more amino acids.

In certain embodiments, the second medium comprises one or more of the components as substantially as described in Table 2 or Table B herein.

In certain embodiments, the second medium is substantially as described in Table 2 or Table B herein.

In certain embodiments the culture medium meets the physiological and nutritional requirements of gametes or embryos and also minimizes intracellular stress such as metabolic stress, pH stress, osmotic stress and oxidative stress.

In one embodiment the culture medium mimics the in vivo conditions appropriate for the developmental stage of the gamete or embryo.

In one embodiment there is provided a first medium which mimics the composition of compounds found in the oviduct.

In one embodiment there is provided a first medium which supports the zygote and cleavage stage embryo.

In one embodiment the first medium comprises pyruvate at a higher concentration than the second medium.

In one embodiment the first medium comprises pyruvate at a similar concentration to that found in the oviduct (e.g. of a pregnant female). The pyruvate concentration in the oviduct is typically about 0.32 mM.

In one embodiment the concentration of pyruvate in the first medium is about 0.1-1.0 mM, for example about 0.32 mM.

In one embodiment the first medium comprises lactate at a higher concentration than the second medium.

In one embodiment the first medium comprises lactate at a similar concentration to that found in the oviduct (e.g. of a pregnant female). The lactate concentration in the oviduct is typically about 10.5 mM.

In one embodiment the concentration of lactate in the first medium is about 5-20 mM, for example about 10.5 mM.

In one embodiment the first medium comprises glucose at a lower concentration than the second medium. The primary energy source at the pre-compaction stage is pyruvate and lactate. In one embodiment the first medium comprises glucose at a similar concentration to that found in the oviduct (e.g. of a pregnant female). The glucose concentration in the oviduct is typically 0.5 mM.

In one embodiment the concentration of glucose in the first medium is about 0.05-5 mM, e.g. about 0.5 mM.

In one embodiment the first medium comprises non-essential amino acids (NEAA).

In one embodiment there is provided a second or compounding medium which in addition to the first medium provides a compounded medium which supports blastocyst development and differentiation.

In one embodiment there is provided a compounded medium which mimics the composition of compounds found in the uterus (e.g. of a pregnant female).

In one embodiment the second medium comprises glucose at a higher concentration than the first medium. After compaction the embryo has high oxidative capacity.

In one embodiment the concentration of glucose in the second medium is about 200%, 300%, 400%, 500%, 600% or 700% of the concentration of glucose in the first medium.

In one embodiment the second medium comprises about 1-11 mM glucose, e.g. about 5.8 mM glucose.

In one embodiment the second medium comprises pyruvate at a lower concentration than the first medium. In one embodiment the compounded medium comprises pyruvate at a similar concentration to that found in the uterus (e.g. of a pregnant female). In one embodiment the concentration of pyruvate in the second medium is about 10%, 20%, 30%, 40% or 50%, of the concentration of pyruvate in the first medium. In one embodiment the concentration of pyruvate in the second medium is about 0 to 1.0 mM. In one embodiment the second medium comprises no or substantially no pyruvate.

In one embodiment the second medium comprises lactate at a lower concentration than the first medium. In one embodiment the compounded medium comprises lactate at a similar concentration to that found in the uterus (e.g. of a pregnant female). In one embodiment the concentration of lactate in the second medium is about 10%, 20%, 30%, 40%, 50%, or 60% of the concentration of lactate in the first medium. In one embodiment the concentration of lactate in the second medium is about 0.1-10 mM, e.g. preferably about 1.24 mM or less.

In one embodiment the second medium comprises both essential and non-essential amino acids.

In one embodiment there is provided a compounded medium which supports the post-compacted embryo.

In one embodiment the compounded medium comprises pyruvate at a similar concentration to that found in the uterus (e.g. of a pregnant female). The pyruvate concentration in the uterus is typically about 0.10 mM.

In one embodiment the concentration of pyruvate in the compounded medium is about 0.05-1 mM e.g. preferably less than or equal to 0.25 mM.

In one embodiment the compounded medium comprises lactate at a similar concentration to that found in the uterus. The lactate concentration in the uterus is about 5-6 mM, such as about 5.87 mM.

In one embodiment the concentration of lactate in the compounded medium is about 2.55-15 mM e.g. about 5 mM, e.g. about 5.25-5.87 mM.

In one embodiment the compounded medium comprises glucose at a similar concentration to that found in the uterus. The glucose concentration in the uterus is about 3 mM, e.g. about 3.15 mM.

In one embodiment the concentration of glucose in the compounded medium is about 0.53-8, e.g. about 3 mM, e.g. about 3.15 mM.

Essential amino acids include cysteine, histidine, isoleucine, leucine, lysine, methionine, valine, arginine, glutamine, phenylalanine, threonine and/or tryptophan. In one embodiment the amino acid may be a non-essential amino acid, such as proline, serine, alanine, asparagine, aspartic acid, glycine and/or glutamic acid. Media that support the development of zygotes up to 8-cells may typically be supplemented with non-essential amino acids, such as proline, serine, alanine, asparagine, aspartic acid, glycine and/or glutamic acid. Media that support the development of 8-cell embryos up to the blastocyst stage are typically supplemented with essential amino acids, such cysteine, histidine, isoleucine, leucine, lysine, methionine, valine, arginine, glutamine, phenylalanine, threonine and/or tryptophan.

In certain embodiments, the incubating of the embryo in the compounded embryo culture medium comprises a period of time in the range from 24 to 144 hours.

In certain embodiments, the incubating of the embryo in the compounded embryo culture medium comprises a period of time in the range from 24 to 144, 36 to 144, 48 to 144, 60 to 144, 72 to 144, 96 to 144, 120 to 144, 24 to 120, 48 to 120, 60 to 120, 72 to 120, 96 to 120, 24 to 96, 36 to 96, 36 to 96, 48 to 96, 60 to 96, 72 to 96, 24 to 72, 36 to 72, 48 to 72, 60 to 72, 24 to 60, 36 to 60, 48 to 60, 24 to 48, 36 to 48, or 24 to 36 hours.

In certain embodiments, the ammonium concentration throughout culturing is less than 300 μM. In certain embodiments, the ammonium concentration throughout culturing is less than 190 μM. In certain embodiments, the ammonium concentration throughout culturing is less than 150 μM. In certain embodiments, the ammonium concentration throughout culturing is less than 120 μM. In certain embodiments, the ammonium concentration throughout culturing is less than 100 μM. In certain embodiments, the ammonium concentration throughout culturing is less than 60 μM. In certain embodiments, the ammonium concentration throughout culturing is less than 50 μM. In certain embodiments, the ammonium concentration throughout culturing is less than 18 μM.

In certain embodiments, the method does not comprise removing the embryo from the first embryo culture medium and/or washing of the embryo prior to addition of the second medium.

Certain embodiments of the present disclosure provide a non-human embryo for implantation produced according to a method as described herein.

Certain embodiments of the present disclosure provide a method of culturing an embryo for implantation, the method comprising use of a compounding medium added to a first culture medium to culture the embryo.

The first culture medium and the compounding medium (also referred to as a second medium in certain embodiments) are as described herein. Embryos for culturing are as described herein.

Certain embodiments of the present disclosure provide a method of culturing an embryo for implantation, the method comprising culturing the embryo in one or more culture media that does not substantially comprise EDTA.

Culture media that do not substantially comprise EDTA are as described herein.

Certain embodiments of the present disclosure provide a method of culturing an embryo for implantation, the method comprising culturing the embryo in one or more culture media comprising acetyl-carnitine (and/or an acceptable salt and/or derivative thereof).

Culture media comprising acetyl carnitine are as described herein.

Certain embodiments of the present disclosure comprise a method of assisted reproduction, the method comprising culturing an embryo for implantation using a method as described herein and implanting the embryo into a subject.

Examples of assisted reproductive technologies are as described herein. In certain embodiments, the method of assisted reproduction comprises in vitro fertilization.

In certain embodiments, the embryo is a human embryo.

In certain embodiments, the embryo is an animal embryo. In certain embodiments, the embryo is a mammalian embryo such as an embryo from a livestock animal (such as a horse, a cow, a sheep, a goat, a pig, a camel), a domestic animal (such as a dog or a cat) and other types of animals such as non-human primates, rabbits, mice and laboratory animals. Other types of animals are contemplated.

In certain embodiments, the subject is a human subject suffering from, or susceptible to, reduced fertility, a disease or condition associated with dysfunctional ovulation (such as polycystic ovarian syndrome or hyperprolactinemia), damaged fallopian tubes, presence of adhesions, or other disease or condition that results in reduced fertility.

In certain embodiments, the subject is an animal subject. In certain embodiments, the subject is a mammalian subject, such as a livestock animal (such as a horse, a cow, a sheep, a goat, a pig, a camel), a domestic animal (such as a dog or a cat) and other types of animals such as non-human primates, rabbits, mice and laboratory animals. Other types of animals are contemplated.

Methods for implantation/transfer of embryos in human or animal subjects are known in the art, for example “Embryo Transfer” (2008) ed. by Gautam N. Allahbadia, Rubina Merchant, Anshan Publishers, “The Artificial Insemination and Embryo Transfer of Dairy and Beef Cattle (including Information Pertaining to Goats, Sheep, Horses, Swine, and Other Animals): A Handbook and Laboratory Manual” (2004) Ed. by Jere R. Mitchell, Gordon Allen Doak, Pearson/Prentice Hall Publishers, and “Manipulating the Mouse Embryo: A Laboratory Manual” (Oct. 31, 2013) Fourth edition Paperback by Richard Behringer, Marina Gertsenstein, Kristina Nagy and Andras Nagy, Cold Spring Harbour Laboratory Press.

Certain embodiments of the present disclosure provide an embryo culture medium.

In certain embodiments, the embryo culture medium is an embryo culture medium for culturing a pre-compaction embryo. In certain embodiments, the embryo culture medium is an embryo culture medium for culturing a post-compaction embryo.

Certain embodiments of the present disclosure provide an embryo culture medium comprising acetyl-carnitine (and/or an acceptable salt and/or derivative thereof).

Certain embodiments of the present disclosure provide an embryo culture medium comprising substantially no EDTA.

Certain embodiments of the present disclosure provide an embryo culture medium comprising acetyl-carnitine (and/or an acceptable salt and/or derivative thereof) and substantially no EDTA. In certain embodiments, the embryo culture medium is a culture medium for a human embryo. In certain embodiments, the embryo culture medium is a culture medium for a human subject suffering from, or susceptible to, reduced fertility, a disease or condition associated with dysfunctional ovulation (such as polycystic ovarian syndrome or hyperprolactinemia), damaged fallopian tubes, presence of adhesions, or other disease or condition that results in reduced fertility.

In certain embodiments, the embryo culture medium is an embryo culture medium for an animal embryo. In certain embodiments, the embryo culture medium is an embryo culture medium for a mammalian embryo such as an embryo from a livestock animal (such as a horse, a cow, a sheep, a goat, a pig, a camel), a domestic animal (such as a dog or a cat) and other types of animals such as non-human primates, rabbits, mice and laboratory animals. Other types of animals are contemplated.

In certain embodiments, the embryo culture medium is an embryo culture medium for use in an assisted reproductive technology. Examples of assisted reproductive technologies include in vitro fertilization (IVF), in vitro maturation of oocytes, intracytoplasmic sperm injection (ICSI) and cytoplasmic transfer.

In certain embodiments, the embryo culture medium comprises a culture medium for a pre-compaction embryo. Culture media for a pre-compaction embryo are as described herein.

In certain embodiments, the embryo culture medium does not comprise EDTA. In certain embodiments, the embryo culture medium does not substantially comprise EDTA.

In certain embodiments, the embryo culture medium comprises acetyl-carnitine, and/or an acceptable salt and/or derivative thereof. In certain embodiments, the embryo culture medium comprises an effective amount of acetyl carnitine (and/or an acceptable salt and/or derivative thereof). In certain embodiments, the embryo culture medium comprises a concentration of acetyl carnitine comprising 0.04 mM to 1 mM.

In certain embodiments, the embryo culture medium comprises pyruvate, such a sodium pyruvate. In certain embodiments, the embryo culture medium comprises an effective amount of pyruvate. In certain embodiments, the embryo culture medium comprises greater than 0.32 mM pyruvate.

In certain embodiments, the embryo culture medium comprises aspartate/aspartic acid. In certain embodiments, the fembryo culture medium comprises an effective amount of aspartate. In certain embodiments, the embryo culture medium comprises greater than 0.32 mM aspartate.

In certain embodiments, the embryo culture medium comprises glycine/glycinate. In certain embodiments, the embryo culture medium comprises an effective amount of glycine. In certain embodiments, the embryo culture medium comprises greater than 0.32 mM glycine.

In certain embodiments, the embryo culture medium comprises one or more of the following components: at least 0.1 mM glucose; at least 2 mM lactate, at least 0.1 mM pyruvate; 0.12 mM acetyl-carnitine, 0.1 mM aspartate, 0.1 mM glycine and at least 0.1 mM glutamine and/or alanyl-glutamine, and/or about one or more of the aforementioned concentrations.

In certain embodiments, the embryo culture medium comprises one or more of the components as substantially as described in Table 1 herein.

In certain embodiments, the embryo medium is substantially as described in Table 1 herein.

In certain embodiments, the embryo culture medium is a human embryo culture medium. In certain embodiments, the embryo culture medium is an animal embryo culture medium.

Certain embodiments of the present disclosure provide an embryo culture medium comprising pyruvate and/or lactate and substantially no ethylenediaminetetraacetic acid (EDTA) and/or a salt thereof.

In certain embodiments, embryo culture medium comprises greater than 0.32 mM pyruvate.

In certain embodiments, the embryo culture medium comprises greater than 0.32 mM aspartate.

In certain embodiments, the embryo culture medium comprises greater than 0.32 mM glycine.

In certain embodiments, the embryo culture medium comprises a concentration of acetyl carnitine of 0.04 mM to 1 mM.

In certain embodiments, the embryo culture medium comprises one orof more of the following components: at 1 mM glucose, 1 mM lactate, 0.10 mM pyruvate, 0.01 mM acetyl-carnitine, 0.01 mM aspartate, 0.05 mM glycine and at least 0.1 mM glutamine and/or alanyl-glutamine.

In certain embodiments, the embryo culture medium is a human embryo culture medium. In certain embodiments, the embryo culture medium is an animal embryo culture medium.

In certain embodiments, the embryo culture medium comprises one or more of the components as substantially as described in Table 1 herein.

In certain embodiments, the embryo medium is substantially as described in Table 1 herein.

Certain embodiments of the present disclosure provide a method of culturing an embryo, the method comprising culturing the embryo in a medium as described herein.

Certain embodiments of the present disclosure provide a compounding medium for addition to an embryo culture medium.

Certain embodiments of the present disclosure provide a compounding medium for addition to an embryo culture medium, the medium comprising a glucose concentration of at least 3.5 mM glucose.

Certain embodiments of the present disclosure provide a compounding medium for addition to an embryo culture medium, the medium comprising a glucose concentration of at least 3.5 mM glucose and less lactate or pyruvate than the first embryo culture medium.

In some embodiments the compounding medium comprises about 10%, 20%, 30%, 40%, 50%, 60%, 70%, 80%, or 90% of the lactate or pyruvate content compared to the first embryo culture medium. In certain embodiments, the compounding medium comprises no or substantially no pyruvate and less than 10 mM lactate, less than 5 mM lactate, less than or equal to 1.24 mM lactate.

In certain embodiments, the compounding medium is to be used for producing a compounded medium for culturing a post-compaction embryo.

In certain embodiments, the compounding medium comprises less acetyl-carnitine or EDTA than the first embryo culture medium. In some embodiments the second medium comprises about 10%, 20%, 30%, 40%, 50%, 60%, 70%, 80%, or 90% of the acetyl-carnitine or EDTA content compared to the first embryo culture medium. In certain embodiments, the compounding medium comprises substantially no acetyl-carnitine (and/or an acceptable salt and/or a derivative thereof) or EDTA.

In certain embodiments, the compounding medium comprises less glutamine and/or alanyl-glutamine than the first embryo culture medium. In some embodiments the second medium comprises about 10%, 20%, 30%, 40%, 50%, 60%, 70%, 80%, or 90% of the glutamine and/or alanyl-glutamine content compared to the first embryo culture medium. In certain embodiments, the second medium comprises less than 0.5 mM glutamine or 0.1 mM or less glutamine or no or substantially no glutamine and/or less than 3 mM alanyl-glutamine or 1.5 mM or less alanyl-glutamine, or no or substantially no alanyl-glutamine.

In certain embodiments, the compounding medium comprises one or more of the following components: at least 3.5 mM glucose, at least 0.06 mM aspartate, and substantially no glycine.

In certain embodiments, the compounding medium comprises one or more of the components as substantially as described in Table 2 or Table B herein.

In certain embodiments, the compounding medium is substantially as described in Table 2 or Table B herein.

Certain embodiments of the present disclosure provide a compounded medium as described herein.

Certain embodiments of the present disclosure provide a compounded medium comprising acetyl carnitine and/or an acceptable salt and/or derivative thereof.

Certain embodiments of the present disclosure provide a compounded medium comprising acetyl carnitine (and/or an acceptable salt and/or derivative thereof) and no or substantially no EDTA. The term “substantially no EDTA” as used herein in relation to the compounded means 0.01 mM or less EDTA.

In one embodiment the compounded medium comprises about 0.1 mM acetyl cartinine.

Certain embodiments of the present disclosure provide a compounded medium comprising acetyl carnitine (and/or an acceptable salt and/or derivative thereof) and comprising substantially no EDTA.

Certain embodiments of the present disclosure provide an embryo culture medium for culturing a post-compaction embryo, the embryo culture medium comprising acetyl carnitine, and/or an acceptable salt and/or derivative thereof.

Certain embodiments of the present disclosure provide an embryo culture medium for culturing a post-compaction embryo, the embryo culture medium comprising substantially no EDTA.

Certain embodiments of the present disclosure provide an embryo culture medium for culturing a post-compaction embryo, the embryo culture medium comprising acetyl-carnitine (and/or an acceptable salt and/or derivative thereof) and comprising substantially no EDTA.

Certain embodiments of the present disclosure provide a medium comprising one or more of the components as described for the compounded medium in Table 2 herein. Certain embodiments of the present disclosure provide a medium substantially as described in Table 2 or Table C herein.

Certain embodiments of the present disclosure provide a compounded medium comprising one or more of the components as described for the compounded medium in Table 2 or Table C herein.

Certain embodiments of the present disclosure provide a compounded medium substantially as described in Table 2 or Table C herein.

Certain embodiments of the present disclosure provide a compounded medium comprising glucose at a concentration of about 0.5 mM to 8 mM or a concentration of about 2 mM to 7 mM a concentration of about 3 mM to 6 mM or a concentration of about 3 mM to 4 mM. In one embodiment the compounded medium comprises glucose at a concentration of about 3 mM.

Certain embodiments of the present disclosure provide a compounded medium comprising pyruvate at a concentration of about 0.05 to 1.00 mM or a concentration of about 0.10 to 0.9 mM or a concentration of about 0.1 to 0.5 mM or a concentration of about 0.1 to 0.3 mM, or a concentration of about 0.15 to 0.3 mM. In one embodiment the compounded medium comprises pyruvate at a concentration of about 0.16 mM to 0.25 mM.

Certain embodiments of the present disclosure provide a compounded medium comprising lactate at a concentration of about 2.5 mM to 15.0 mM or a concentration of about 5 mM to 12.5 mM or a concentration of about 5 mM to 7 mM or a concentration of about 5 mM to 6 mM. In one embodiment the compounded medium comprises lactate at a concentration of about 0.25 to 5.87 mM.

Certain embodiments of the present disclosure provide a compounded medium comprising non-essential amino acids.

Certain embodiments of the present disclosure provide a compounded medium comprising essential amino acids.

Certain embodiments of the present disclosure provide a compounded medium comprising both non essential amino acids and essential amino acids.

In some embodiments ammonium levels are maintained under 190 μM for the entire culture period. In another embodiment ammonium levels are maintained under 150 μM for the entire culture period. In another embodiment ammonium levels are maintained under 120 μM for the entire culture period. In one embodiment ammonium levels are maintained under 100 μM for the entire culture period. In one embodiment the ammonium levels are maintained under 60 μM for the entire culture period. In one embodiment the ammonium levels are maintained or under 18.5 μM for the entire culture period.

In certain embodiments the ammonium levels in the first embryo culture medium are maintained under 190 μM, under 150 μM, under 120 μM, under 100 μM, under 60 μM or under 18.5 μM. In one embodiment the ammonium levels in the first embryo culture medium are maintained under 190 μM. In one embodiment the ammonium levels in the first embryo culture medium are maintained under 150 μM. In one embodiment the ammonium levels in the first embryo culture medium are maintained under 120 μM. In one embodiment the ammonium levels in the first embryo culture medium are maintained under 100 μM. In one embodiment the ammonium levels in the first embryo culture medium are maintained under 60 μM. In one embodiment the ammonium levels in the first embryo culture medium are maintained under 18.5 μM.

In certain embodiments the ammonium levels in the compounded medium is maintained under 190 μM, under 150 μM, under 12 μM 0, under 100 μM, under 60 μM or under 18.5 μM. In one embodiment the ammonium levels in the compounded medium are maintained under 190 μM. In one embodiment the ammonium levels in the compounded medium are maintained under 150 μM. In one embodiment the ammonium levels in the compounded medium are maintained under 120 μM. In one embodiment the ammonium levels in the compounded medium are maintained under 100 μM. In one embodiment the ammonium levels in the compounded medium are maintained under 60 μM. In one embodiment the ammonium levels in the compounded medium are maintained under 18.5 μM.

Certain embodiments of the present disclosure provide a method of culturing an embryo for implantation, the method comprising:
incubating a pre-compaction embryo in an embryo culture medium as described herein;
adding a compounding medium as described herein to the first embryo culture medium to form a compounded embryo culture medium; and
further incubating the embryo in the compounded embryo culture medium for implantation.

Certain embodiments of the present disclosure provide a combination product.

Certain embodiments of the present disclosure provide a combination product comprising:
(i) a first embryo culture medium as described herein; and
(ii) a compounding medium as described herein.

In certain embodiments, the combination product further comprises one or more of (a) instructions for culturing an embryo in the embryo culture medium; (b) instructions for adding the compounding medium to the embryo culture medium to form a compounded embryo culture medium; (c) instructions for incubating the embryo in the compounded embryo culture medium; and (d) instructions for implantation of the embryo.

Certain embodiments of the present disclosure provide a kit for performing a method as described herein.

Certain embodiments of the present disclosure provide a kit comprising:
(i) an embryo culture medium as described herein; and/or
(ii) a compounding medium as described herein.

Certain embodiments of the present disclosure provide a non-human animal produced using a method of assisted reproduction as described herein.

Certain embodiments of the present disclosure provide a method of vitrification of an embryo.

Certain embodiments of the method of vitrification of an embryo, the method comprising:
incubating a pre-compaction stage embryo in a first embryo culture medium;
adding a second medium to the first embryo culture medium to form a compounded embryo culture medium;
further incubating the embryo in the compounded embryo culture medium; and
freezing the embryo.

Methods for embryo vitrification are known in the art.

Certain embodiments of the present disclosure provide a vitrified embryo produced using a method of vitrification as described herein.

Methods of determining embryo quality in vitro are known in the art. Any method may be used for determining the quality of an embryo. Methods for assessing the quality of an embryo include the measurement of total cell number and measurement of differentiation into trophectoderm (TE) and inner cell mass (ICM). The term “inner cell mass (ICM)” is herein defined is the mass of cells inside the embryo that will eventually give rise to the definitive structures of the fetus. Inner cell mass may be also known as the embryoblast or pluriblast.

The term “trophectoderm (TE)” is herein defined as the cells forming the outer layer of a blastocyst, e.g. which provide nutrients to the embryo and develop into a large part of the placenta. The blastocyst scoring system developed by Gardener et al. (Blastocyst score affects implantation and pregnancy outcome: towards a single blastocyst transfer. Fertil Steril. 2000; 73(6):1155-1158) incorporated herein by reference, may be used to calculate a quality score for a blastocyst. Kinetic markers and morphological markers of human embryo quality can be measured using time-lapse recordings.

Standard techniques may be used for recombinant DNA technology, oligonucleotide synthesis, antibody production, peptide synthesis, tissue culture and transfection. Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), herein incorporated by reference.

In certain embodiments, the present disclosure is described in further detail in the following numbered paragraphs:

1. A method of culturing an embryo for implantation, the method comprising:
incubating a pre-compaction stage embryo in a first embryo culture medium;
adding a second medium to the first embryo culture medium to form a compounded embryo culture medium; and
further incubating the embryo in the compounded embryo culture medium for implantation.
   2. The method according to paragraph 1, wherein the incubating of the pre-compaction embryo in the first embryo culture medium comprises a period of time sufficient for the embryo to form a post-compaction embryo.
   3. The method according to paragraphs 1 or 2, wherein the incubating of the pre-compaction embryo in the first embryo culture medium comprises a period of time in the range from 24 to 72 hours.
   4. The method according to any one of paragraphs 1 to 3, wherein the incubating of the embryo in the compounded embryo culture medium comprises a period of time sufficient for the embryo to form a morula or blastocyst.
   5. The method according to any one of paragraphs 1 to 4, wherein the incubating of the embryo in the compounded embryo culture medium comprises a period of time in the range from 24 to 144 hours.
   6. The method according to any one of paragraphs 1 to 5, wherein the first embryo culture medium does not comprise EDTA.
   7. The method according to any one of paragraphs 1 to 6, wherein the first embryo culture medium comprises acetyl-carnitine and/or an acceptable salt and/or derivative thereof.
   8. The method according to paragraph 7, wherein the concentration of acetyl carnitine comprises 0.04 mM to 1 mM.
   9. The method according to any one of paragraphs 1 to 8, wherein the first embryo culture medium comprises greater than 0.32 mM pyruvate.
   10. The method according to any one of paragraphs 1 to 9, wherein the first embryo culture medium comprises greater than 0.32 mM aspartate.
   11. The method according to any one of paragraphs 1 to 10, wherein the first embryo culture medium comprises greater than 0.32 mM glycine.
   12. The method according to any one of paragraphs 1 to 11, wherein the first embryo culture medium comprises one of more of the following components: at least 0.1 mM glucose; at least 2 mM lactate, at least 01 mM pyruvate; 0.12 mM acetyl-carnitine, 0.1 mM aspartate, 0.1 mM glycine and at least 0.1 mM glutamine and/or alanyl-glutamine.
   13. The method according to any one of paragraphs 1 to 12, wherein the second medium comprises substantially no pyruvate or lactate.
   14. The method according to any one of paragraphs 1 to 13, wherein the second medium comprises substantially no acetyl-carnitine or EDTA.
   15. The method according to any one of paragraphs 1 to 14, wherein the second medium comprises substantially no glutamine and/or alanyl-glutamine.
   16. The method according to any one of paragraphs 1 to 15, wherein the second medium comprises a glucose concentration of at least 1 mM glucose.
   17. The method according to any one of paragraphs 1 to 16, wherein the second medium comprises one of more of the following components: at least 1 mM glucose, at least 0.01 mM aspartate, and substantially no glycine.
   18. The method according to any one of paragraphs 1 to 17, wherein the compounded embryo culture medium comprises one of more of the following components: at least 1 mM glucose, 1 mM lactate, 0.10 mM pyruvate, 0.01 mM acetyl-carnitine, 0.01 mM aspartate, 0.05 mM glycine and at least 0.1 mM glutamine and/or alanyl-glutamine.
   19. The method according to any one of paragraphs 1 to 18, wherein the ammonium concentration throughout culturing is less than 300 μM.
   20. The method according to any one of paragraphs 1 to 19, wherein the method does not comprise removing the embryo from the first embryo culture medium and/or washing of the embryo prior to addition of the second medium.
   21. The method according to any one of paragraphs 1 to 20, wherein the embryo is a human embryo.
   22. The method according to any one of paragraphs 1 to 20, wherein the embryo is an animal embryo.
   22. A method of culturing an embryo for implantation, the method comprising use of a compounding medium added to a first culture medium to culture the embryo.
   23. A method of culturing an embryo for implantation, the method comprising culturing the embryo in one or more culture media that does not substantially comprise EDTA.
   24. A method of culturing an embryo for implantation, the method comprising culturing the embryo in one or more culture media comprising acetyl-carnitine, and/or an acceptable salt and/or derivative thereof.
   25. A method of assisted reproduction, the method comprising culturing an embryo for implantation using a method according to any one of paragraphs 1 to 24 and implanting the embryo into a subject.
   26. The method according to paragraph 25, wherein the method of assisted reproductions comprises in vitro fertilization.
   27. The method according to paragraphs 25 or 26, wherein the subject is an animal subject.
   28. The method according to paragraphs 25 or 26, wherein the subject is a human subject.
   29. An embryo culture medium comprising acetyl-carnitine, and/or an acceptable salt and/or derivative thereof.
   30. The embryo culture medium according to paragraph 29, wherein the medium comprises a concentration of acetyl carnitine comprises 0.04 mM to 1 mM
   31. The embryo culture medium according to paragraphs 29 or 30, wherein the medium comprises greater than 0.32 mM pyruvate.
   32. The embryo culture medium according to any one of paragraphs 29 to 31, wherein the medium comprises greater than 0.32 mM aspartate.
   33. The embryo culture medium according to any one of paragraphs 29 to 32, wherein the medium comprises greater than 0.32 mM glycine.
   34. The embryo culture medium according to any one of paragraphs 29 to 33, wherein the medium comprises one of more of the following components: at least 1 mM glucose, 1 mM lactate, 0.10 mM pyruvate, 0.01 mM acetyl-carnitine, 0.01 mM aspartate, 0.05 mM glycine and at least 0.1 mM glutamine and/or alanyl-glutamine.
   35. The embryo culture medium according to any one of paragraphs 29 to 34, wherein the medium does not substantially comprise EDTA.
   36. An embryo culture medium comprising pyruvate and/or lactate and substantially no ethylenediaminetetraacetic acid and/or a salt thereof.
   37. The embryo culture medium according to paragraph 36, wherein the medium comprises greater than 0.32 mM pyruvate.
   38. The embryo according to paragraphs 36 or 37, wherein the medium comprises greater than 0.32 mM aspartate.
   39. The embryo culture medium according to any one of paragraphs 36 to 38, wherein the medium comprises greater than 0.32 mM glycine.
   40. The embryo culture medium according to any one of paragraphs 36 to 39, wherein the medium comprises a concentration of acetyl carnitine of 0.04 mM to 1 mM.
   41. The embryo culture medium according to any one of paragraphs 36 to 40, wherein the medium comprises one of more of the following components: at 1 mM glucose, 1 mM lactate, 0.10 mM pyruvate, 0.01 mM acetyl-carnitine, 0.01 mM aspartate, 0.05 mM glycine and at least 0.1 mM glutamine and/or alanyl-glutamine.
   42. The embryo culture medium according to any one of paragraphs 36 to 41, wherein the embryo culture medium is a human embryo culture medium.
   43. The embryo culture medium according to any one of paragraphs 36 to 41, wherein the embryo culture medium is an animal embryo culture medium.
   44. A method of culturing an embryo, the method comprising culturing the embryo in a medium according to any one of paragraphs 29 to 43.
   45. A compounding medium for addition to an embryo culture medium, the medium comprising a glucose concentration of at least 3.5 mM glucose and substantially no lactate and/or pyruvate.
   46. The compounding medium according to paragraph 45, wherein the medium comprises substantially no acetyl-carnitine or EDTA.
   47. The compounding medium according to paragraphs 45 or 46, wherein the medium comprises substantially no glutamine and/or alanyl-glutamine.
   48. The compounding medium according to any one of paragraphs 45 to 47, wherein the medium comprises one of more of the following components: at least 3.5 mM glucose, at least 0.06 mM aspartate, and substantially no glycine.
   49. A method of culturing an embryo for implantation, the method comprising:
incubating a pre-compaction embryo in an embryo culture medium according to any one of paragraphs 29 to 43;
adding a compounding medium according to any one of paragraphs 45 to 48 to the first embryo culture medium to form a compounded embryo culture medium; and
further incubating the embryo in the compounded embryo culture medium for implantation.
   50. A combination product comprising:
(i) an embryo culture medium according to any one of paragraphs 29 to 43; and
(ii) a compounding medium according to any one of paragraphs 45 to 48.
   51. The combination product according to paragraph 44, wherein the combination product further comprises one or more of (a) instructions for culturing an embryo in the embryo culture medium; (b) instructions for adding the compounding medium to the embryo culture medium to form a compounded embryo culture medium; (c) instructions for incubating the embryo in the compounded embryo culture medium; and (d) instructions for implantation of the embryo.
   52. A kit for performing the method according to any one of paragraphs 1 to 28.
   53. A kit comprising:
(i) an embryo culture medium according to any one of paragraphs 29 to 43; and/or
(ii) a compounding medium according to any one of paragraphs 45 to 48.
   54. A non-human animal produced using the method according to any one of paragraphs 25 to 28.
   55. A method of vitrification of an embryo, the method comprising:
incubating a pre-compaction stage embryo in a first embryo culture medium;
adding a second medium to the first embryo culture medium to form a compounded embryo culture medium;
further incubating the embryo in the compounded embryo culture medium; and
freezing the embryo.
   56. A vitrified embryo produced using the method according to paragraph 55.

The present disclosure is further described by the following examples. It is to be understood that the following description is for the purpose of describing particular embodiments only and is not intended to be limiting with respect to the above description.

### Example 1—Compounding Media

The composition for compounding culture medium 1 (CCM1) is shown in Table 1, showing the amounts of the components for producing 2 litres of the medium and the subsequent concentrations.

TABLE 1

| Chemical | Amount (g) | | mM |
|---|---|---|---|
| NaCl | 10.68 | | 92.07 |
| KCl | 0.82 | | 5.5 |
| MgSO₄ | 0.984 | | 4 |
| NaHCO₃ | 4.204 | | 25 |
| KH₂PO₄ | 0.068 | | 0.25 |
| Glucose | 0.18 | | 0.5 |
| Na Lactate | 2.3523 | | 10.5 |
| Na Pyruvate | 0.11 | | 0.5 |
| Acetyl-carnitine | 0.0812 | | 0.2 |
| Taurine | 0.0312 | | 0.125 |
| Alanine | 0.0178 | | 0.1 |
| Aspartate | 0.1331 | | 0.5 |
| Asparagine | 0.0264 | | 0.1 |
| Glycine | 0.075 | | 0.5 |
| Glutamate | 0.0294 | | 0.1 |
| Proline | 0.023 | | 0.1 |
| Serine | 0.021 | | 0.1 |
| Glycl-glutamine | 20 | ml | 1 |
| CaCl₂ | 0.222 | | 1 |
| Arginine-HCl | | | 0 |
| Cystine | | | 0 |
| Histidine-HCl—H₂O | | | 0 |
| Isoleucine | | | 0 |
| Leucine | | | 0 |
| Lysine | | | 0 |
| Methionine | | | 0 |
| Phenylalanine | | | 0 |
| Threonine | | | 0 |
| Tryptophan | | | 0 |
| Tyrosine | | | 0 |
| Valine | | | 0 |
| D-Ca | 1780 | mls | 0 |

| | |
|---|---|
| Pantothenate | |
| Pyridoxal-HCl | 0 |
| Thiamine-HCl | 0 |
| Riboflavin H₂O | 0 |

The composition for compounding culture medium 2 (CCM2) is as shown in Table 2, showing the amounts of the components for producing 2 litres of the medium and the subsequent concentrations. The last column shows the final concentration of the components when mixed with compounding culture medium 1.

TABLE 2

| | | | | mM |
|---|---|---|---|---|
| | Amount | | | Final |
| Chemical | (g) | | mM | Soln |
| NaCl | 10.68 | | 92.07 | 92.07 |
| KCl | 0.82 | | 5.5 | 5.5 |
| MgSO₄ | 0.984 | | 4 | 4 |
| NaHCO₃ | 4.204 | | 25 | 25 |
| KH₂PO₄ | 0.2041 | | 0.75 | 0.5 |
| Glucose | 2.0898 | | 5.8 | 3.15 |
| Na Lactate | 0 | | 0 | 5.25 |
| Na Pyruvate | 0 | | 0 | 0.25 |
| Acetyl carnitine | 0 | | 0 | 0.1 |
| Taurine | 0 | | 0 | 0.0625 |
| Alanine | 0.0178 | | 0.1 | 0.1 |
| Aspartate | 0.0266 | | 0.1 | 0.1 |
| Asparagine | 0.0264 | | 0.1 | 0.1 |
| Glycine | 0 | | 0 | 0.25 |
| Glutamate | 0.0294 | | 0.1 | 0.1 |
| Proline | 0.023 | | 0.1 | 0.1 |
| Serine | 0.021 | | 0.1 | 0.1 |
| Glycl-glutamine | 0 | | 0 | 0.5 |
| CaCl₂ | 0.222 | | 1 | 1 |
| Arginine-HCl | Amino | | .06 | 0.3 |
| Cystine | Acid | | 0.1 | 0.05 |
| Histidine-HCl—H₂O | Solution | | 0.2 | 0.1 |
| Isoleucine | 40 | mls | 0.4 | 0.2 |
| Leucine | | | 0.4 | 0.2 |
| Lysine | | | 0.4 | 0.2 |
| Methionine | | | 0.1 | 0.05 |
| Phenylalanine | | | 0.2 | 0.1 |
| Threonine | | | 0.4 | 0.2 |
| Tryptophan | | | 0.5 | 0.25 |
| Tyrosine | | | 0.2 | 0.1 |
| Valine | | | 0.4 | 0.2 |
| D-Ca | Vitamin | | | |
| Pantothenate | stock | | | |
| Pyridoxal-HCl | solution | | | |
| Thiamine-HCl | 40 | mls | | |
| Riboflavin H₂O | 1720 | mls | | |

### Materials and Methods:

### (i) Animals and Diet

All mice were obtained and housed by the University of Adelaide Laboratory Animal Services, Adelaide, Australia. The animal ethics committee of the University of Adelaide approved all experiments, and animals were handled in accordance with the Australian code for the care and use of animals for scientific purposes 8th edition (2013). All mice were maintained at 24° C. on a 12 hour light, 12 hour dark illumination cycle with food and water provided ad libitum. C57B16 male mice (8-10 weeks of age) were used as spermatozoa donors. Pre-pubescent CBAF1 female mice (C57B16×CBA) were super-ovulated for experiments.

### (ii) Media Composition

House-made Compound Culture Media (CCM1) and G-1 Plus (Vitrolife, Kungsbacka, Sweden), the composition of G-1 Plus is as described in Table A above, supplemented with Human Serum Albumin. These solutions were diluted to varying concentrations (1 ug/ml, 10 ug/ml, 100 ug/ml, 1000 ug/ml), supplemented with 10% Human Serum Albumin (HSA, Vitrolife)

G-MOPS supplemented with 10% HSA (Vitrolife) was used as the collection and handling medium.

For treatment group 1: House-made CCM was supplemented with 10% HSA.
For treatment group 2: House-made CCM was supplemented with 100 ug/ml 10-HDA and 10% HSA.
For treatment group 3: G-1 Plus and G-2 Plus sequential culture system (Vitrolife) (G-1 Plus and G-2 plus are the equivalent to G-1 and G-2 respectively each pre-supplemented with 10% HSA).
For treatment group 4: Cook Cleavage and Blastocyst media (William A. Cook Australia Pty. Ltd., QLD, Australia).
For treatment group 5: LifeGlobal Global media (LifeGlobal Group, USA) was supplemented with 10% HSA (Vitrolife).

Plastic-ware and consumables used for culture were previously tested for their ability to support embryo development using a 1-cell mouse bioassay (Gardner D L, Lane M. Culture of Mammalian Preimplantation Embryo. “A Labortaory Guide to the Mammalian Embryo.” Ed. D K Gardner, M Lane, A J Watson, p 41-61).

(iii) Embryo Collection and Culture

Pre-pubescent CBAF1 female mice (C57B16×CBA) were super-ovulated by intra-peritoneal (IP) injection of 5 IU of pregnant mare's gonadotrophin (PMSG; Folligon; Intervet, Bendigo, Victoria, Australia). This was followed 46-48 hours later by an IP injection of 5 IU human chorionic gonadotrophin (hCG; Pregnyl; Organon, Oss, Holland). Females were housed with C57B16 males overnight to facilitate mating, which was determined the following morning by the presence of a vaginal plug. Culture media drops were overlayed with Ovoil (Vitrolife) to prevent evaporation of the media and pre-gassed at 37° C., 6% CO₂, 5% O₂ for a minimum of 4 hours. 23.75 hours after hCG injection zygotes were collected into warmed G-MOPS medium supplemented with 10% HSA before incubation with lmg/ml hyaluronidase (Sigma) to initiate denuding. Viable zygotes were washed thoroughly through G-MOPS supplemented with 10% HSA before being randomly allocated into treatment groups. Zygotes in CCM treatment groups were cultured for 44 hours before the pre-gassed secondary compound media was added. All other embryos were transferred into pre-gassed phase 2 media after 44 hours of culture. All treatment groups were incubated for a total of 92 hours.

### (iv) Embryo Development

Fertilisation was indicated by cleavage to the 2-cell stage after 20 hours of culture. On-time embryo assessments were determined for 8-cell development after 44 hours of culture, morula and early blastocyst development after 75 hours of culture and blastocyst expansion and hatching after 92 hours of culture.

### (v) Differential Staining for Blastocyst Cell Number and Allocation

To determine cleavage rates and allocation of cells to the trophectoderm (TE) and inner cell mass (ICM) in the blastocyst, differential staining was performed as previously described (Mitchell M, Bakos H W, Lane M. “Paternal diet-induced obesity impairs embryo development and implantation in the mouse.” Fertil Steril. 2011 March 15; 95(4):1349-53). Blastocysts were placed into 0.5% pronase (Sigma) at 37° C. until the zona pellucida dissolved, then washed in G-MOPS (without HSA, Vitrolife) before incubation in 10 μM of 2,4,6-trinitrobenzenesulfonic acid (TNBS, Sigma) at 4° C. for 10 minutes. Following a second wash, blastocysts were transferred into 0.1 mg/μl anti-dinitrophenyl-BSA (anti-DNP, Sigma) for 10 minutes at 37° C. before a third wash and then placed in guinea pig serum (Sigma) with Propidium Iodide (PI, Sigma) for 5 minutes at 37° C. Finally, embryos were placed in bisbenzimide (Sigma) at 4° C. overnight. The following day the stained embryos were washed through 100% ethanol (Sigma) and mounted in glycerol (Asia Pacific Specialty Chemicals Ltd, Seven Hills, NSW, AUS) on a glass slide to be visualized using a fluorescent microscope under an ultraviolet filter (Olympus BX-51 wavelength emission 350 nm) at ×400 magnification, where TE cells appeared pink and ICM cells appeared blue. The cell nuclei were counted independently and data was reported as an average cell number per blastocyst.

### (vi) Epiblast Staining for Blastocyst Cell Number and Allocation

To determine development rates and allocation of cells to the epiblast and ICM, epiblast staining was performed as previously described. On day 6 of development blastocysts were fixed in 4% paraformaldehyde (Sigma) overnight before being stored in PVP/PBS (Sigma) the following day. Blastocysts were neutralised in 0.1M glycine in PBS (Sigma), washed in 0.25% Triton/PBS (Sigma) and incubated in 1° Ab mixture (1:200 Nanog (M-149) Rabbit polyclonal IgG sc33760 and 1:100 Oct 3/4 (N-19) Goat Polyclonal IgG sc-8628 (Santa Cruz Biotechnology, Texas, USA)) at 37° C. for 1.5 hours. Blastocysts were washed twice before incubation in 2° Ab mixture (1:100 Alexa Fluor® 488 Donkey Anti-Rabbit IgG (H+L) Antibody A-21206 and 1:100 Alexa Fluor® 594 Donkey Anti-Goat IgG (H+L) A-11058 Antibody (Life Technologies, California, USA)) at room temperature for 2 hours. Blastocysts were washed twice more before being incubated in Hoescht in PBS (Sigma) for 2-5 minutes. Embryos were mounted in a small drop of glycerol on a glass slide with paraffin spacers and cover slipped. Stained embryos were visualized using a fluorescent microscope (Olympus U-MWU2 wavelength emission 400 nm) at ×400 magnification. ICM and epiblast cells were counted under the appropriate filters before the total cell number was counted under UV and these were reported as an average per blastocyst.

(vii) Vitrification and Warming

Vitrification was performed at 37° C. on day 5 blastocysts using solutions containing propandiol, sucrose, ethylene glycol and Ficoll. Blastocysts were placed into G-MOPS+10% HSA for 5 minutes and then moved into equilibration solution for 2 minutes (G-MOPS+10% with 8% propandiol and 8% ethylene glycol). Blastocysts were then placed in a 20 μl drop of vitrification solution (GMOPS+10% with 16% propandiol and 16% ethylene glycol, 0.65 M sucrose, 10 mg/ml Ficoll), pipetted up and down 3 times before being placed onto a Rapid-I (Vitrolife) in groups of 6-10 in approximately 300 nl of fluid, inserted into Rapid-I Straw (Vitrolife), sealed and plunged into liquid nitrogen within 45 seconds.

Warming of the vitrified blastocysts will be performed at 37° C. in solutions containing decreasing concentrations of sucrose. To warm, the outer Rapid-I straw will be cut and the inner straw containing the blastocysts will be removed from liquid nitrogen and immediately immersed into G-MOPS+10% and 0.65M sucrose. The blastocysts will be removed within 30 seconds and placed into GMOPS+10% and 0.325M sucrose for 1 min, then transferred to GMOPS+10% and 0.125M sucrose for 2 min before being transferred into G-MOPS+10% for 5 min. Blastocysts will then be washed 3 times through G-2 Plus and placed into culture until required for transfer.

### Example 2—In Vitro Culture Procedure

Prior to culture: mix 9 ml CCM 1 with HSA (1 ml of 100 mg/ml HSA) and mix 9 ml CCM 2 with HSA (1 ml of 100 mg/ml HSA).

### Step 1:

1. Either the night before or in the morning, label the underside and lid of dish.
2. Pre-rinse a sterile 10 μl pipette tip by taking up CCM 1 media and expelling/discarding before making your drops.
3. Place drops of media in pairs to provide one 10 μl wash drop and one 10 μl culture drop each.
4. Cover with Embryo-tested Oil
5. Place dishes in the incubator at 5-6% CO₂ and +37° C. preferably with reduced oxygen.
6. Collect oviducts and denude the putative zygotes.
7. Rinse embryos through their wash drop in the culture dish and place 4-12 (dependent on species) embryos in each culture drop for development to the 8-cell stage (around 48 h of culture).

### Step 2:

8. Place CCM 2 medium into incubator at 5-6% CO₂ and +37° C. preferably with reduced oxygen either the night before or in the morning
9. With a fresh, pre-rinsed tip each time, add 10 μl of the warmed and gassed CCM2 to each of the 10 μl drops of CCM 1 which has embryos in it. To make a total of 20 μl drops.
10. Return dishes to incubator and culture to the blastocyst stage (24-48 h of culture).

### Example 3—Embryo Development Using Compound Culture Media

Mouse embryos were cultured in the different culture systems as described in Example 2. The effect of the different culture conditions on embryo development is shown in Table 3.

CCM is an illustrative compound culture system of the present disclosure. Vitrolife, and Sydney IVF are conventional, sequential culture systems which are commercially available (Treatment Groups 3-5 of Example 2). Global/GLOBAL is an example of a mono culture media, wherein after 3 days of culture, it is advised to replace the media with fresh media with the same composition.

TABLE 3

| Embryo Development | CCM | Vitrolife | Sydney IVF | Global/GLOBAL |
|---|---|---|---|---|
| 78 h of culture (%) | | | | |
| Total Blast | 70.3^{ab} | 83.8^{b} | 83.6^{b} | 60.7^{a} |
| Early Blast | 13.0 | 22.8 | 23.5 | 27.7 |
| Expanded Blast | 57.2^{a} | 61.1^{a} | 60.1^{a} | 33.0^{bc} |
| 96 h of culture (%) | | | | |
| Total Blast | 81.1 | 92.9 | 91.0 | 89.8 |
| Expanded Blast | 17.3^{a} | 17.0^{a} | 12.5^{a} | 35.5^{b} |
| Hatching/Hatched Blast | 59.8^{ab} | 71.2^{b} | 77.4^{b} | 39.7^{c} |

Percentage of cleavage embryos was similar between treatment groups. Post cleavage embryo development is expressed as percentage of the number of cleaved embryos per group. Date is expressed at average±SEM. Different letters denote significant difference at P<0.05. N<200 embryos per treatment group.

This demonstrates that CCM supports developmental rates to the blastocyst stage at equivalent or superior to current products on the market.

The effect of the different culture conditions on blastocyst cell number is shown in Table 4.

TABLE 4

| Blastocyst | | | | |
|---|---|---|---|---|
| cell number | | Sydney | | Global/ |
| (96 h culture) | CCM | IVF | Vitrolife | GLOBAL |
| TCN | 91.0 ± 2.4^{a} | 94.7 ± 2.6^{a} | 79.4 ± 2.3^{b} | 73.3 ± 2.8^{b} |
| TE Cell Number | 71.3 ± 2.0^{ab} | 75.6 ± 2.2^{a} | 63.1 ± 2.0^{bc} | 57.4 ± 2.3^{c} |
| ICM Cell Number | 19.7 ± 0.6^{a} | 19.2 ± 0.7^{a} | 16.3 ± 0.6^{b} | 15.9 ± 0.7^{b} |
| TE Cell Number | 78.3 ± 0.6^{ab} | 79.4 ± 0.6^{b} | 79.2 ± 0.5^{b} | 77.9 ± 0.6^{ab} |
| (% of TCN) | | | | |
| ICM Cell Number | 21.7 ± 0.5 | 20.6 ± 0.6 | 20.8 ± 0.6 | 22.1 ± 0.7 |
| (% TCN) | | | | |

TCN-total cell number, TE-trophectoderm, ICM-inner cell mass. N < 80 embryos per treatment group. Data is expressed at average ± SEM. Different letters denote significant difference at P < 0.05.

Assessment of total cell number and differentiation into ICM and TE are used as in vitro measures of embryo quality. This demonstrates that CCM supports blastocyst quality at equivalent or superior to current products on the market

The culture system developed provides for the dynamic nature of the developing embryo however it eliminates the need for a media change thus improving on current formulations. The system involves culturing the embryo in an initial medium formulation which contains, amongst other components, increased levels of pyruvate (0.5 mM), aspartate (0.5 mM), glycine (0.5 mM), acetyl-carnitine (0.2 mM) but no EDTA compared to standard media formulations.

After incubation in the first medium, a second medium is added to the original drop to alter the medium requirement of the later stage embryos. This medium lacks a variety of components such as pyruvate, lactate and glutamine in order to generate a decreasing gradient, whilst it contains higher concentrations of glucose, essential amino acids and vitamins to create an increasing gradient.

The culture system developed provides a number of important benefits over existing systems, including one or more of eliminating the need for embryos to be handled and removed from the original media, eliminating the need to remove the embryos from their original culture medium, eliminating the need to wash embryos during culturing, increasing cell number in the embryo at the blastocyst stage, e.g. when measured at 5 to 6 days of culturing in a system according to the present invention preserving the gradient of pyruvate concentration, preserving the gradient of lactate concentration, preserving gradient of glucose concentration, maintaining the switch from non-essential to all 20 amino acids, maintaining ammonium levels <18.5μ M for the entire culture period, via the use of amino acid stable di-peptides, and eliminates the requirement for EDTA in the culture medium, while maintaining mitochondrial function.

### Example 4—In Vitro Culture Procedure for Human Embryos

Prior to culture: mix 9 ml CCM 1 with HSA (1 ml of 100 mg/ml HSA) and mix 9 ml CCM 2 with HSA (1 ml of 100 mg/ml HSA).

### Step 1:

1. Either the night before or in the morning, label the underside and lid of dish
2. Pre-rinse a sterile 10 μl pipette tip by taking up CCM 1 media and expelling/discarding before making the drops.
3. Place drops of media in pairs to provide one 20-25 μl wash drop and one 20-25 μl culture drop each.
4. Cover with Embryo-tested Oil
5. Place dishes in the incubator at 5-6% CO₂ and 37° C. preferably with reduced oxygen.
6. Collect oviducts and denude the putative zygotes.
7. Rinse embryos through their wash drop in the culture dish and place 1-5 embryos in each culture drop for development to the 8-cell stage (around 48 h of culture).

### Step 2:

8. Place CCM 2 medium into the in the incubator at 5-6% CO₂ and 37° C. preferably with reduced oxygen either the night before or in the morning
9. With a fresh, pre-rinsed tip each time, add 20-25 μl of the warmed and gassed CCM2 to each of the 20-25 μl drops of CCM 1 which has embryos in it. To make a total of 40-50 μl drops.
10. Return dishes to incubator and culture to the morula/blastocyst stage (24-96 h of culture).

### Example 5—Implantation of Embryos

The culture media system as described herein provides morula and blastocyst stage embryos that are suitable for transfer to a recipient. These embryos are suitable to be replaced in either non-surgical or surgical procedures. In each case the recipient will be prepared by either the administration of medications or by monitoring of the natural ovulatory cycle. For medicated cycles, embryos could then be returned to the reproductive tract before or during the time period that the uterine lining is deemed to be receptive to embryos and in a natural cycle between the time period of ovulation and implantation windows.

Embryos can be drawn up into any device that is used for embryo transfer and can be replaced into either the oviduct or uterus either non-surgically (through cervix, with or without ultrasound guidance) or surgically by either exposing the tract and then inserting the transfer device or by puncture of the reproductive tract and insertion of the transfer device.

Methods for implantation are known in the art and are described herein.

### Example 6—Products

The compounding culture system may, for example, be provided by products as follows:

### Liquid Media

(i) Aseptic Compounding Culture Medium 1 (with or without HSA) provided in a sterile bottle;
(ii) Aseptic Compounding Culture Medium 2 (with or without HSA) provided in a sterile bottle; and
optionally any one or more of the following:
HSA (dissolved in suitable solvent for example at 100 mg/ml) or lyophilised;
instructions for culturing an embryo in the CCM1;
instructions for adding the CCM2 to CCM1 to form a compounded embryo culture medium;
instructions for incubating an embryo in the compounded embryo culture medium;
instructions for implantation of an embryo.
For animal applications, other sources of albumin may be used, such as bovine serum albumin.

### Lyophilised

(i) Lyophilised Compounding Culture Medium 1 (with or without HSA) provided in a sterile container;
(ii) Lyophilised Compounding Culture Medium 2 (with or without HSA) provided in a sterile bottle; and
optionally any one or more of the following:
Diluent (water—sterile filtered, or other suitable buffer/ionic solution)
HSA, lyophilised or dissolved in suitable solvent (for example at 100 mg/ml);
instructions for reconstituting media from lyophilised components;
instructions for culturing an embryo in the CCM1;
instructions for adding the CCM2 to CCM1 to form a compounded embryo culture medium;
instructions for incubating an embryo in the compounded embryo culture medium;
instructions for implantation of an embryo.
For animal applications, other sources of albumin may be used, such as bovine serum albumin.

### Example 7—Embryo Development Using Compound Culture Media

All human embryo culture media have been developed using the mouse as a model because of the similarities in embryo metabolism/physiology and the fact that there are well established mouse embryo development/viability parameters.

### Materials and Methods:

This study was designed to compare mouse embryo development in 2 different media systems: Sequential media (G-1™/G-2™) and G-Compounding media (formulation given in Table B above).

The complete formulation for G-1™ is provided in Table A (above).

The complete formulation for the G-2™ medium is provided in the table below:

| | Most | | | |
|---|---|---|---|---|
| | Preferred | | Preferred | |
| Component | Concentration | | Range | |
| NaCl | 90.08 | | 80.0-100 | |
| KCl | 5.5 | | 3.5-7.5 | |
| NaH₂PO₄ | 0.25 | | 0.05-1.5 | |
| MgSO₄ | 1 | | 0.2-4.0 | |
| NaHCO₃ | 2.5 | | 15-30.0 | |
| CaCl₂ | 1.8 | | 0.8-2.8 | |
| Glucose | 3.15 | | 0.5-5.5 | |
| NaLactate (L-isomer) | 5.87 | | 2.0-20.0 | |
| NaPyruvate | 0.1 | | 0.01-1.0 | |
| Alanine | 0.1 | | 0.01-0.5 | |
| Asparate | 0.1 | | 0.01-0.5 | |
| Asparagine | 0.1 | | 0.01-0.5 | |
| Glutamate | 0.1 | | 0.01-0.5 | |
| Alanyl-Glutamine | 1 | | 0.01-2.0 | |
| Glycine | 0.1 | | 0.01-0.5 | |
| Proline | 0.1 | | 0.01-0.5 | |
| Serine | 0.1 | | 0.01-0.5 | |
| L-Arginine HCl | 0.6 | | 01-1.2 | |
| L-Cystine 2HCl | 0.1 | | 0.05-0.25 | |
| L-Histidine-HCl—H2O | 0.2 | | 0.1-0.4 | |
| L-Isoleucine | 0.4 | | 0.1-0.8 | |
| L-Leucine | 0.4 | | 0.1-0.8 | |
| L-Lysine-HCl | 0.4 | | 0.1-0.8 | |
| L-Methionine | 0.1 | | 0.05-0.25 | |
| L-Phenylalanine | 0.2 | | 0.1-0.4 | |
| L-Threonine | 0.4 | | 0.1-0.8 | |
| L-Tryptophan | 0.5 | | 0.1-0.9 | |
| L-Tyrosine 2Na | 0.2 | | 0.1-0.4 | |
| L-Valine | 0.4 | | 0.1-0.8 | |
| D-Ca Pantothenate | 0.002 | | 0.001-0.004 | |
| Choline Chloride | 0.007 | | 0.003-0.01 | |
| Folic Acid | 0.0023 | | 0.001-0.0045 | |
| i-Inositol | 0.0111 | | 0.005-0.02 | |
| Niacinamide | 0.0082 | | 0.004-0.016 | |
| Pyridoxal HCl | 0.0049 | | 0.002-0.01 | |
| Riboflavin | 0.0003 | | 0.0001-0.0006 | |
| Thiamine HCl | 0.003 | | 0.001-0.006 | |
| HSA | 5 | mg/ml | 1-10 | mg/ml |
| Hyaluronate | 0.1 | mg/ml | 0.02-0.5 | mg/ml |

The concentrations in the above table are provided in mM, unless otherwise indicated. F1 hybrid females were superovulated and ovulation triggered. The female mice were mated to F1 hybrid male mice and housed together overnight. The following day, each female was evaluated for the presence of a copulation plug. 1-cell mouse embryos were collected from female mice 22 hours following trigger of ovulation. This was considered Day 1 of embryo development. The embryos were pooled and then divided between the 2 treatment groups; G-1™/G-2™ and G-Compounding media. All media contained 5 mg/ml of human serum albumin. Embryos were cultured in 10 μl drops of media (10 embryos per drop) in standard culture dishes at 37° C. (7.3% CO₂ and ambient O₂).

For the G-1™/G-2™ group, embryos were cultured in G-1™ from the morning of Day 1 until the morning of Day 3. On Day 3, the embryos were moved to a new pre-equilibrated culture dish containing G-2™ and cultured until the morning of Day 5.

For the G-Compounding media group embryos were cultured in G-1™ from the morning of Day 1 until the morning of Day 3. On Day 3, G-Compounding media was added to the G-1™ media to create a 1:1 ratio of G-1™ to G-Compounding media and the embryos cultured until the morning of Day 5. The G-compounding media media was pre-equilibrated in the original culture dish from Day 1 until its use on Day 3.

Embryo morphology was evaluated on the afternoon of day 4 (78 hours) and the morning of day 5 (96 hours), then embryos were fixed, stained and the number of cells in each blastocyst counted. Morphology results are presented as the mean of arcsine transformed percentages and standard deviations. Blastocyst cell numbers are presented as the mean cell number and the 95% confidence interval. Statistical comparisons were determined for morphology and blastocyst cell number by Fisher's Exact test and Student's T-test, respectively.

Briefly, 1-cell mouse embryos were collected from female mice following mating. This is considered Day 1 of embryo development. The embryos were pooled and then divided between the sequential media treatment (Vitrolife G-1™ followed by G-2™) and the compound media treatment (Vitrolife G-1™ with G-compounding media subsequently added). As per the standard protocol, embryos were scored on the afternoon of Day 4 and/or the morning of Day 5. Following the Day 5 score, embryos were fixed, stained and the number of cells in each blastocyst counted.

### Results:

### Embryo Development Using G-1™/G-2™ and G-Compounding Media

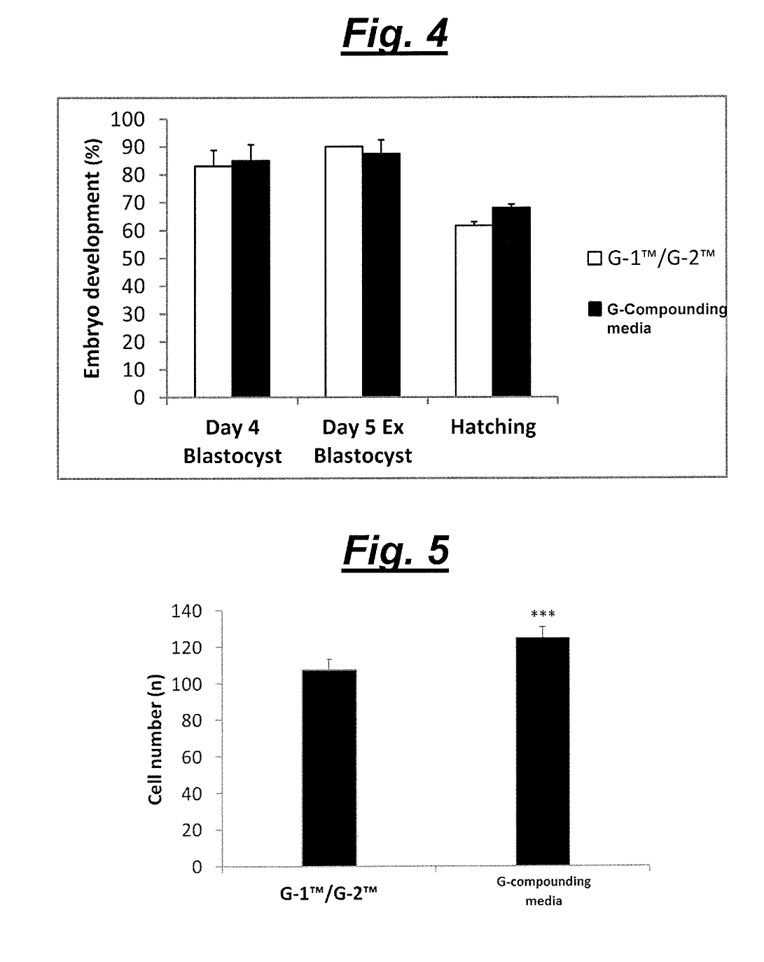

Four replicates were performed with 120 embryos in each group. FIG. 4 shows embryo development of mouse embryos in sequential G-1™/G-2™ treatment compared to G-1™ with G-compounding media subsequently added. No statistical difference (p=>0.2) was observed in the percentage of embryos reaching the blastocyst stage on Day 4, the expanding blastocyst stage on Day 5 or the percentage of blastocysts that were hatching. FIG. 5 shows the comparison of cell number. There is a significant increase (p=<0.0001) in mean number of cells that composed the Day 5 blastocysts when the embryos had been cultured in G-1™ with G-compounding media subsequently added compared to sequential G-1™/G-2™.

### CONCLUSIONS

These data provide evidence that the G-compounding media medium supports mouse embryo development at least equivalent to the Sequential media G-1™/G-2™. Embryo development to the blastocyst stage on Day 4, expanding blastocyst stage on Day 5 and the rates of hatching with G-compounding media were parallel to G-1™/G-2™. The number of cells in a Day 5 blastocyst was significantly higher in embryos cultured in G-compounding media. Because embryo cell number is an indicator of viability this data set suggests that G-compounding media can support embryo development and perhaps even increase viability compared to G-1™/G-2™.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The term “culture” as used herein may mean maintaining in conditions suitable for embryo growth and/or maturation.

Growth rate can be measured, for example, by measuring the trophectoderm cell number, inner cell mass number, or total cell number, for example when measured at 5 days of culturing in a culture system of the present invention (i.e. culture in a first embryo culture medium followed by the addition of a second culture medium at about day 3). Suitably, the growth rate is measured by measuring the total cell number at 5 days of culturing in a culture system of the present invention.

The preimplantation period of embryo development varies between mammalian species. However, the embryos of humans and mice share the most similar length of development (around 4 to 5 days) and also exhibit quite similar implantation. The blastocysts of both species reach a similar size and the nutrient utilization patterns are very similar between mice the embryos of humans and mice (see for example Gott A L, Hardy K, Winston R M, Leese H J. Non-invasive measurement of pyruvate and glucose uptake and lactate production by single human preimplantation embryos. Hum Reprod. 1990; 5(1):104-8; Leese H J, Barton A M. Pyruvate and glucose uptake by mouse ova and preimplantation embryos. J Reprod Fertil. 1984; 72(1):9-13; and Gardner D K, Wale P L. Analysis of metabolism to select viable human embryos for transfer. Fertility and Sterility. 2013; 99(4):1062-72). The similarities shared between human and mouse development are such that they allow for the evaluation of human development from experiments performed on murine systems (see Csaba Pribensky et al. Reproductive Biomedicine Online (2010) 20, 371-379). Consequently, it is held that the mouse makes the most appropriate model for the human preimplantation embryo.

An embryo is approximately spherical and is composed of one or more cells (blastomeres) surrounded by the acellular matrix known as the zona pellucida. The zona pellucida performs a variety of functions until the embryo hatches, and is a good landmark for embryo evaluation. The zona pellucida is spherical and translucent, and should be clearly distinguishable from cellular debris.

During embryonic development, blastomere numbers increase geometrically (1-2-4-8-16- etc.). Synchronous cell cleavage is generally maintained to the 8-cell stage in human embryos. After that, cell cleavage becomes asynchronous and finally individual cells possess their own cell cycle. Human embryos produced during infertility treatment are usually transferred to the recipient before 8-blastomere stage. In some cases human embryos are also cultivated to the blastocyst stage before transfer. This is preferably done when many good quality embryos are available or prolonged incubation is necessary to await the result of a pre-implantation genetic diagnosis (PGD). However, there is a tendency towards prolonged incubation as the incubation technology improves.

Accordingly, the term embryo is used in the following to denote each of the stages fertilized oocyte, zygote, 2-cell, 4-cell, 8-cell, 16-cell, compaction, morula, blastocyst, expanded blastocyst and hatched blastocyst, as well as all stages in between (e.g. 3-cell or 5-cell).

An embryo is formed when an oocyte is fertilized by fusion or injection of a sperm cell (spermatozoa). The term is traditionally used also after hatching (i.e. rupture of zona pelucida) and the ensuing implantation. For humans the fertilized oocyte is traditionally called a zygote or an embryo for the first 8 weeks. After that (i.e. after eight weeks and when all major organs have been formed) it is called a fetus. However the distinction between zygote, embryo and fetus is not generally well defined. The terms embryo and zygote are used herein interchangeably.

In one embodiment, the embryo may be cultured individually.

In yet another embodiment, the embryo is cultured in the compound culture system of the present disclosure until the blastocyst stage, expanded blastocyst stage or hatched blastocyst stage.

A reference to an embryo as referred to herein include both a singular embryo and multiple embryos. In other word an embryo means “an embryo or embryos”.

A reference to a stem cell as referred to herein include both a singular stem cell and multiple stem cells. In other word a stem cell means “a stem cell or stem cells”.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

The term “about” or “approximately” means an acceptable error for a particular value, which depends in part on how the value is measured or determined. In certain embodiments, “about” can mean 1 or more standard deviations. When the antecedent term “about” is applied to a recited range or value it denotes an approximation within the deviation in the range or value known or expected in the art from the measurements method.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1. A method of culturing an embryo for implantation, the method comprising:
incubating a pre-compaction stage embryo in a first embryo culture medium;
adding a second medium to the first embryo culture medium to form a compounded embryo culture medium; and
further incubating the embryo in the compounded embryo culture medium for implantation.

2. The method according to claim 1, wherein the incubating of the pre-compaction embryo in the first embryo culture medium comprises a period of time sufficient for the embryo to form a post-compaction embryo.

3. The method according to claim 1 or 2, wherein the incubating of the pre-compaction embryo in the first embryo culture medium comprises a period of time in the range from 24 to 72 hours.

4. The method according to any one of claims 1 to 3, wherein the incubating of the embryo in the compounded embryo culture medium comprises a period of time sufficient for the embryo to form a morula or blastocyst.

5. The method according to any one of claims 1 to 4, wherein the incubating of the embryo in the compounded embryo culture medium comprises a period of time in the range from 24 to 144 hours.

6. The method according to any one of claims 1 to 5, wherein the first or second medium or compounded medium comprises one or more antioxidants.

7. The method according to any one of claims 1 to 6, wherein the first or second medium or compounded medium comprises one or more antioxidants selected from the group comprising: acetyl-carnitine, acetyl cysteine or lipoic acid and/or an acceptable salt and/or derivative thereof.

8. The method according to claim 7, wherein the concentration of acetyl carnitine in the first or second medium or compounded medium comprises 5 μM to 1 mM.

9. The method according to any one of claims 1 to 8, wherein the first embryo culture medium comprises greater than 0.1 mM pyruvate, preferably greater than 0.20 mM pyruvate, preferably greater than 0.25 mM pyruvate, preferably greater than 0.30 mM pyruvate, more preferably equal to or greater than 0.32 mM pyruvate.

10. The method according to any one of claims 1 to 9, wherein the first embryo culture medium comprises greater than 0.01 mM aspartate, preferably greater than 0.10 mM aspartate, preferably greater than 0.15 mM, preferably greater than 0.20 mM, preferably greater than 0.25 mM, preferably greater than 0.30 mM, more preferably equal to or greater than 0.32 mM aspartate.

11. The method according to any one of claims 1 to 10, wherein the first embryo culture medium comprises greater than 0.01 mM glycine, preferably greater than 0.10 mM glycine, preferably greater than 0.15 mM, preferably greater than 0.20 mM, preferably greater than 0.25 mM, preferably greater than 0.30 mM, more preferably equal to or greater than 0.32 mM glycine.

12. The method according to any one of claims 1 to 11, wherein the first embryo culture medium comprises one of more of the following components: at least 0.05 mM glucose, preferably at least 0.1 mM glucose; at least 2 mM lactate, preferably greater than 5 mM lactate; at least 0.1 mM pyruvate, preferably at least 0.3 mM pyruvate; at least 0.01 mM aspartate, preferably at least 0.1 mM aspartate; at least 0.01 mM glycine, preferably at least 0.1 mM glycine; and/or at least 0.1 mM glutamine and/or at least 0.1 mM alanyl-glutamine; and optionally at least 0.12 mM acetyl-carnitine.

13. The method according to any one of claims 1 to 12, wherein the second medium comprises less pyruvate or lactate than the first embryo culture medium, preferably about 10%, 20%, 30%, 40%, 50%, 60%, 70%, 80%, or 90% of the pyruvate or lactate content compared to the first embryo culture medium, more preferably the second medium comprises no pyruvate or less than 10 mM lactate, preferably less than 5 mM lactate, preferably less than or equal to 1.24 mM lactate.

14. The method according to any one of claims 1 to 13, wherein the second medium comprises less acetyl-carnitine or EDTA than the first embryo culture medium, preferably about 10%, 20%, 30%, 40%, 50%, 60%, 70%, 80%, or 90% of the acetyl-carnitine or EDTA content compared to the first embryo culture medium, more preferably the second medium comprises no or substantially no acetyl-carnitine or EDTA.

15. The method according to any one of claims 1 to 14, wherein the second medium comprises less glutamine and/or alanyl-glutamine than the first embryo culture medium, preferably the second medium comprises about 10%, 20%, 30%, 40%, 50%, 60%, 70%, 80%, or 90% of the glutamine and/or alanyl-glutamine content compared to the first embryo culture medium, preferably the second medium comprises less than 0.5 mM glutamine, preferably 0.1 mM or less glutamine, preferably no or substantially no glutamine and/or less than 3 mM alanyl-glutamine, preferably less than 1.5 mM alanyl-glutamine, preferably no or substantially no alanyl-glutamine.

16. The method according to any one of claims 1 to 15, wherein the second medium comprises a glucose concentration of at least 1 mM glucose, preferably at least 5 mM glucose.

17. The method according to any one of claims 1 to 16, wherein the second medium comprises one or more of the following components: at least 1 mM glucose, at least 0.01 mM aspartate, and no or less than 0.5 mM glycine, preferably less than 0.1 mM glycine.

18. The method according to any one of claims 1 to 17, wherein the compounded embryo culture medium comprises one or more of the following components: at least 0.5 mM glucose, preferably at least 3 mM glucose; less than 1 5 mM aspartate, preferably less than 6 mM lactate; less than 1.0 mM pyruvate, preferably less than 0.2 mM pyruvate; at least 0.01 mM aspartate; and at least 0.01 glycine, preferably at least 0.05 mM glycine; and optionally at least 0.06 mM glutamine, preferably at least 0.1 mM glutamine and/or optionally at least 0.06 mM alanyl-glutamine, preferably at least alanyl-glutamine and/or optionally at least 0.01 mM acetyl-carnitine.

19. The method according to any one of claims 1 to 18, wherein the ammonium concentration throughout culturing is less than 300 μM, preferably less than 190 μM.

20. The method according to any one of claims 1 to 19, wherein the method does not comprise removing the embryo from the first embryo culture medium and/or washing of the embryo prior to addition of the second medium.

21. The method according to any one of claims 1 to 20, wherein the embryo is a human embryo.

22. The method according to any one of claims 1 to 20, wherein the embryo is an animal embryo.

23. A method of culturing an embryo for implantation, the method comprising use of a compounding medium added to a first culture medium to culture the embryo.

24. A method of culturing an embryo for implantation, the method comprising culturing the embryo in one or more culture media comprising acetyl-carnitine, and/or an acceptable salt and/or derivative thereof.

25. A method of assisted reproduction, the method comprising culturing an embryo for implantation using a method according to any one of claims 1 to 24 and implanting the embryo into a subject.

26. The method according to claim 25, wherein the method of assisted reproductions comprises in vitro fertilization.

27. The method according to claim 25 or 26, wherein the subject is an animal subject.

28. The method according to claim 25 or 26, wherein the subject is a human subject.

29. A compounding medium for addition to an embryo culture medium, the medium comprising a glucose concentration of at least 1 mM glucose, preferably at least 3.5 mM glucose; and 10 mM or less lactate, preferably 1.24 mM or less lactate; and/or 2 mM or less pyruvate, preferably 0.16 mM or less pyruvate; preferably the medium comprises no or substantially no lactate and/or pyruvate.

30. The compounding medium according to claim 29, wherein the medium comprises 0.01 mM or less acetyl-carnitine or 0.01 mM or less EDTA, preferably the medium comprises substantially no acetyl-carnitine or EDTA.

31. The compounding medium according to claim 29 or 30, wherein the medium comprises less than 0.01 mM glutamine or less than 3 mM, preferably 1.5 mM or less alanyl-glutamine, preferably the medium comprises no or substantially no glutamine and/or alanyl-glutamine.

32. The compounding medium according to any one of claims 29 to 31, wherein the medium comprises one or more of the following components: at least 3.5 mM glucose; at least 0.01 mM aspartate, preferably at least 0.06 mM aspartate; and 0.5 mM or less, preferably 0.1 mM or less glycine; and preferably the medium comprises no or substantially no glycine.

33. A method of culturing an embryo for implantation, the method comprising:
incubating a pre-compaction embryo in a first embryo culture medium;
adding a compounding medium according to any one of claims 29 to 32 to the first embryo culture medium to form a compounded embryo culture medium; and
further incubating the embryo in the compounded embryo culture medium for implantation.

34. A combination product comprising:
(i) a first embryo culture medium; and
(ii) a compounding medium according to any one of claims 29 to 32.

35. The combination product according to claim 34, wherein the combination product further comprises one or more of (a) instructions for culturing an embryo in the embryo culture medium; (b) instructions for adding the compounding medium to the embryo culture medium to form a compounded embryo culture medium; (c) instructions for incubating the embryo in the compounded embryo culture medium; and (d) instructions for implantation of the embryo.

36. A kit for performing the method according to any one of claims 1 to 28.

37. A kit comprising:
(i) a first embryo culture medium; and
(ii) a compounding medium according to any one of claims 29 to 32.

38. A non-human animal produced using the method according to any one of claims 25 to 28.

39. A method of vitrification of an embryo, the method comprising:
incubating a pre-compaction stage embryo in a first embryo culture medium;
adding a second medium to the first embryo culture medium to form a compounded embryo culture medium;
further incubating the embryo in the compounded embryo culture medium; and
freezing the embryo.

40. A vitrified embryo produced using the method according to claim 39.
